(19) United States
(12) Patent Application Publication
(10) Pub. No.: US 2011/0117193 A1
(43) Pub. Date: May 19, 2011

(54) ANTIRETROVIRAL DRUG FORMULATIONS FOR TREATMENT OF CHILDREN EXPOSED TO HIV/AIDS

(75) Inventors: Moji C. Adeyeye, Pittsburgh, PA (US); Fredrick Esseku, Pittsburgh, PA (US); Anajali Joshi, Pittsburgh, PA (US)

(73) Assignee: Duquesne University of the Holy Spirit, Pittsburgh, PA (US)

(21) Appl. No.: 12/863,111

(22) PCT Filed: Jan. 16, 2009
(86) PCT No.: PCT/US09/31285

§ 371 Date: Jan. 11, 2011

Related U.S. Application Data
(60) Provisional application No. 61/021,790, filed on Jan. 17, 2008.

(51) Int. Cl. A61K 9/22; A61K 9/00; A61K 31/7072; A61P 31/18

(52) U.S. Cl. 424/465; 424/484; 514/50

(57) ABSTRACT

The present disclosure provides fast disintegrating formulations for the treatment of human immunodeficiency virus (HIV) and acquired immune deficiency syndrome (AIDS) in patients such as neonatal, perinatal and pediatric children. Neonatal and perinatal formulations provide for the prevention or reduction of incidence of mother to child transmission of HIV. Also provided are formulations and methods for treating pediatric children having HIV/AIDS. The orally administered fast disintegrating formulations are in granule and tablet form and are specially formulation for children to increase adherence to treatment protocols.

### TECHNICAL FIELD

The present disclosure is directed to pharmaceutical formulations useful for reducing the incidence of mother-to-child transmission of the human immunodeficiency virus (HIV) and for the treatment of acquired immune deficiency syndrome (AIDS) in children. The pharmaceutical formulations are rapidly dissolving or reconstitutable and palatable to children, therefore easing problems associated with administration of certain antiretroviral drugs to children. These problems can be particularly challenging in resource-poor countries where potable water is limited. Methods of reducing incidence of mother-to-child transmission of HIV and for treating HIV/AIDS are also disclosed.

### BACKGROUND

Acquired immune deficiency syndrome (AIDS) is a set of symptoms and diseases resulting from damage to the immune system due to human immunodeficiency virus (HIV) infection. The condition progressively reduces the effectiveness of the immune system and leaves the patient susceptible to opportunistic infections and diseases.

The HIV virus is believed to have originated in sub-Saharan Africa during the twentieth century. AIDS was first recognized by the United States Centers for Disease Control and Prevention in 1981 and the HIV virus first identified as its cause in the early 1980's. HIV is a retrovirus that primarily infects vital elements of the human immune system, such as CD4⁺ T cells, macrophages and dendritic cells. Acute HIV infection progresses over time to clinical latent HIV infection, then to early symptomatic HIV infection and finally to AIDS. In the absence of therapy, the median time of progression from HIV infection to AIDS is nine to ten years and the median survival time after developing AIDS is 9.2 months. Due to the ability of the virus to rapidly mutate and develop resistance to certain therapies, the development of a vaccine or cure for HIV/AIDS has proven complicated.

In sub-Saharan Africa, the AIDS epidemic has taken a particularly devastating toll, with over 42 million people estimated to have contracted HIV and an estimated 22.5 million people currently living with HIV/AIDS. At least eight African countries have an HIV/AIDS infection rate among adults that exceeded 15 percent of the population in 2005. According to the United Nations AIDS, an estimated 1.7 million adults and children in sub-Saharan Africa became infected with HIV during the year 2007. In addition, an estimated 1.6 million people died from AIDS related illnesses in Africa in 2007. Due, at least in part, to economic reasons, access to drugs for treating and prevention of HIV/AIDS in Africa has lagged behind the developed world.

While there is no vaccine or cure for HIV/AIDS, treatments involving antiretroviral drugs can slow the course of the disease. Current treatment of HIV infection consists of highly active antiretroviral therapy (HAART). Because of the ability of the virus to develop resistance to one or more effective antiretroviral drugs, certain HAART treatments for adult patients involve combinations (or “cocktails”) of three or more drugs. With HAART treatment, those infected with HIV/AIDS have significantly longer life expectancies than untreated patients.

Transmission of the virus typically occurs via direct contact of a mucous membrane or the bloodstream with a bodily fluid from a person infected with HIV. Since HIV/AIDS has traditionally been transmitted via unprotected sexual acts, sharing of contaminated hypodermic needles among intravenous drug users, or blood transfusions using blood from infected donors, patients suffering from HIV/AIDS are typically adults. As a result HIV/AIDS treatment protocols and formulations are designed and developed for adult physiologies.

However, children can be infected with HIV not only via traditional contact with bodily fluid but also via perinatal transmission. For example, the transmission of the virus from an infected mother to the child can occur in utero, during the last weeks of pregnancy, or during childbirth. The risk of mother to child transmission (MTCT) of the virus may be influenced by the viral load of the mother during childbirth, and certain procedures such as caesarean section may reduce the incidence of transmission. Breastfeeding may also increase the risk of MTCT by about 4%. In the developed world, MTCT is typically addressed by suppression of the virus in the mother. Since children make up only a small percent of the HIV/AIDS infected population, drug therapies, treatment protocols, and formulations have not been targeted toward the prevention and treatment of HIV/AIDS in children. However, in certain third world countries and sub-Saharan Africa, the number of children exposed to and/or infected by HIV is greater, due in part, to socio-economic reasons and a general lack of access to modern medical care and effective antiretroviral drugs to treat infected women during pregnancy. Thus, there is a need for new formulations, treatment protocols, and therapies to prevent MTCT of HIV and to treat children infected with HIV/AIDS.

### BRIEF DESCRIPTION

The various embodiments of the present invention relate to pharmaceutical formulations for the prevention of mother to child transmission of HIV or treatment of HIV/AIDS in children. The formulations are specially developed to address the disease in children and to be readily administered to children. In addition, the formulations are designed for effective treatment of HIV/AIDS in children in undeveloped countries where access to medical treatment and monitoring, and potable water is limited.

According to one embodiment, the present disclosure provides a rapidly dissolving pharmaceutical formulation to reduce the incidence of mother to child transmission of human immunodeficiency virus. The formulation comprises a therapeutically effecting amount of an active medicament selected from zidovudine and nevirapine, and a non-active ingredients matrix. The non-active ingredients matrix comprises one or more excipient comprising from 15% to 95% by weight of the total formulation, one or more superdisintegrant comprising from 1% to 8% by weight of the total formulation, one or more sweeteners comprising from 0.1% to 11% by weight of the total formulation, and one or more lubricants comprising from 0.1% to 3% by weight of the total formulation. The pharmaceutical formulation substantially dissolves in an aqueous solution in less than 60 seconds or even less than 40 seconds or in some cases less than 30 seconds. The pharmaceutical formulation may be in a form of a rapidly dissolving granule formulation or a rapidly dissolving tablet formulation.

In another embodiment, the present disclosure provides for the use of a pharmaceutical formulation in the manufacture of a rapidly dissolving formulation for reducing the incidence of mother to child transmission of human immunodeficiency virus (HIV). The pharmaceutical formulation may be in a form of a rapidly dissolving granule formulation or a rapidly dissolving tablet formulation and comprises a therapeutically effective amount of an active medicament selected from zidovudine and nevirapine, and a non-active ingredients matrix, such as those described in detail herein. The pharmaceutical formulation may be administered to a patient ranging from 0 months to 18 months in age.

In a further embodiment, the present disclosure provides a rapidly dissolving pharmaceutical formulation for the treatment of human immunodeficiency virus/acquired immune deficiency syndrome (HIV/AIDS) in children. The formulation comprises lamivudine in from 0.4% to 10% by weight of the total formulation, zidovudine in from 0.65% to 25% by weight of the total formulation, and a non-active ingredients matrix. The non-active ingredients matrix comprises one or more excipient comprising from 1.0% to 55% by weight of the total formulation, one or more superdisintegrant comprising from 0% to 8% by weight of the total formulation, one or more sweeteners comprising from 0.75% to 92.52% by weight of the total formulation, one or more flavorant comprising from 0% to 0.20% by weight of the total formulation, one or more lubricants comprising from 0% to 3% by weight of the total formulation, and one or more parabens preservative comprising from 0% to 0.44% by weight of the total formulation. In specific embodiments, the pharmaceutical formulation may further comprise nevirapine in from 0.5% to 20% by weight of the total formulation. The pharmaceutical formulation substantially dissolves in an aqueous solution in less than 60 seconds. The pharmaceutical formulation may be in a form of a rapidly dissolving reconstitutable granule formulation or a rapidly dissolving tablet formulation. The present disclosure also provides for a stable aqueous suspension of the reconstitutable pharmaceutical granule formulation of some of the embodiments disclosed herein.

In still another embodiment, the present disclosure provides for the use of a pharmaceutical formulation in the manufacture of a rapidly dissolving formulation for treatment of human immunodeficiency virus (HIV) or acquired immune deficiency syndrome (AIDS) in children. The pharmaceutical formulation may be in a form of a rapidly dissolving granule formulation or a rapidly dissolving tablet formulation and comprises lamivudine in from 0.4% to 10% by weight of the total formulation, zidovudine in from 0.65% to 25% by weight of the total formulation, and a non-active ingredients matrix, such as those described in detail herein. In specific embodiments, the pharmaceutical formulation may further comprise nevirapine in from 0.5% to 20% by weight of the total formulation. The pharmaceutical formulation may be administered to a patient ranging from 3 months to 16 years in age. The pharmaceutical formulation may be administered orally to the patient in the form of a tablet, granules, or as a reconstituted aqueous suspension made from the granules.

### BRIEF DESCRIPTION OF THE DRAWINGS

The disclosure of the present application will be better understood when read in conjunction with the following drawings and figures.

FIG. 1 illustrates the dissolution profile of one embodiment of the zidovudine granule formulation for reducing MTCT transmission of HIV.

FIG. 2 illustrates the dissolution profile of one embodiment of the nevirapine granule formulation for reducing MTCT transmission of HIV.

FIG. 3 illustrates the dissolution profile of one embodiment of the zidovudine tablet formulation for reducing MTCT transmission of HIV.

FIG. 4 illustrates the dissolution profile of one embodiment of the nevirapine tablet formulation for reducing MTCT transmission of HIV.

FIG. 5 illustrates the dissolution profile of one embodiment of the 2 in 1 zidovudine/lamivudine tablet formulation for treating HIV/AIDS in children.

FIG. 6 illustrates the dissolution profile of one embodiment of the 3 in 1 zidovudine/lamivudine/nevirapine tablet formulation for treating HIV/AIDS in children.

FIG. 7 illustrates the dissolution profile of one embodiment of the 2 in 1 zidovudine/lamivudine granule formulation for treating HIV/AIDS in children.

FIG. 8 illustrates the dissolution profile of one embodiment of the 3 in 1 zidovudine/lamivudine/nevirapine granule formulation for treating HIV/AIDS in children.

FIG. 9 illustrates the potency over time of the lamivudine in a suspension of the 2 in 1 zidovudine/lamivudine granule formulation.

FIG. 10 illustrates the potency over time of the zidovudine in a suspension of the 2 in 1 zidovudine/lamivudine granule formulation.

### DETAILED DESCRIPTION

The present disclosure is directed to pharmaceutical formulations useful for reducing the incidence of mother-to-child transmission of the human immunodeficiency virus (HIV) and for the treating HIV infection and acquired immune deficiency syndrome (AIDS) in children. The pharmaceutical formulations are rapidly dissolving and palatable to children. These will therefore ease problems associated with administration of certain antiretroviral drugs to children or for children or older patients who have difficulty swallowing or who cannot swallow. Antiretroviral formulations for HIV/AIDS have not targeted children for a variety of reasons, including: children make up a small percentage of the infected population so most development efforts have focused on adult patients; difficulty in developing children formulations due to regulatory issues, difficulty and cost of clinical trials, and potential litigation issues among others. The present disclosure presents novel and inventive pharmaceutical formulations and methods for treating children exposed to HIV/AIDS.

Other than the operating examples, or where otherwise indicated, all numbers expressing quantities of ingredients, processing conditions and the like used in the specification and claims are to be understood as being modified in all instances by the term “about”. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical values, however, may contain certain errors, such as, for example, equipment and/or operator error, necessarily resulting from the standard deviation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of less than or equal to 10.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The present disclosure describes several different features and aspects of the invention with reference to various exemplary non-limiting embodiments. It is understood, however, that the invention embraces numerous alternative embodiments, which may be accomplished by combining any of the different features, aspects, and embodiments described herein in any combination that one of ordinary skill in the art would find useful.

In the developed world, MTCT is typically addressed by suppression of the virus in the mother or by limiting the child's exposure to the various bodily fluids during childbirth, for example, by utilizing caesarian section birth. However, in addition to potential exposure to HIV in utero and during birth, newborns may also face continued exposure to HIV through breastfeeding. Thus, an alternative approach to reduce the incidence of MTCT of HIV is to administer a single entity antiretroviral drug to the newborn. While antiretroviral drug “cocktails” may be used to treat adult patients with HIV/AIDS, such drug combinations may not be necessary or desirable for preventing MTCT of the virus in perinatal infants. First, the maturity of the virus in the newborn is typically less than in infected adults. That is, the virus in perinatal children has not has time or opportunity to mutate and develop drug resistance. Thus, treatment with single antiretroviral entities may effectively reduce the MTCT without need to rely on multi-component antiretroviral cocktails. Further, significant toxicities are associated with antiretroviral drug cocktails, which may cause undesired side effects upon administration to the developing physiology of the perinatal or infant. However, treating perinatal and other infants with smaller dosages of adult versions of the antiretroviral drugs is not an effective alternative due, for example, to accurate dosing, dispensing, and palatability issues. According to one embodiment, the present disclosure provides for rapidly dissolving pharmaceutical formulations to reduce the incidence of mother to child transmission (MTCT) of HIV.

The rapidly dissolving pharmaceutical formulation to reduce the incidence of mother to child transmission of HIV may comprise a therapeutically effective amount of an active antiretroviral medicament selected from zidovudine and nevirapine, and a non-active ingredients matrix, wherein the formulation substantially dissolves in an aqueous solution in less than 60 seconds, and in certain embodiments less than 40 seconds or even less than 30 seconds. The non-active ingredients matrix may comprise one or more excipient comprising from 15% to 95% by weight of the total formulation, one or more superdisintegrant comprising from 1% to 8% by weight of the total formulation, one or more sweetener comprising from 0.1% to 11% by weight of the total formulation, and one or more lubricants comprising from 0.1% to 3% by weight of the total formulation. As used herein, the term “substantially dissolves” means that the formulation is greater than 80% dissolved, solubilized or suspended in the solvent after the designated time.

The formulations of the present embodiments are designed and formulated to be administered to infants up to 18 months in age to prevent or reduce the incidence of MTCT of HIV. The formulations are designed to rapidly dissolve so that they may be readily placed and dissolve on the tongue of the infant or dissolved in an aqueous solution, such as formula, milk, pudding, applesauce, or other readily consumable food product prior to feeding to the infant. Rapid dissolution is important to prevent rejection of the formulation by the patient. In addition, the formulations are designed to be highly palatable to the patient. The antiretroviral drugs described herein may have a highly bitter, unpleasant taste which may increase rejection (spitting out) by the patient who will not understand the necessity of the drug. Consequently, the pharmaceutical formulations described in the various embodiments herein have been carefully prepared with one or more sweeteners to counter or mask the unpleasant taste of the drug and to ease administration. Simple addition of sweeteners to the formulations is not straightforward since the precise combination of sweeteners must be established to maximize palatability. In addition to sweeteners, flavorants (such as, for example, cherry, grape, raspberry, bubble gum, mixed fruit or other flavorants which may be natural and/or artificial flavorants) may also be incorporated into the various formulation to increase palatability.

Zidovudine (also know as azidothymidine (AZT), 1-[(2R,4S,5S)-4-azido-5-(hydroxymethyl)oxolan-2-yl]-5-methyl-1,2,3,4-tetrahydropyrimidine-2,4-dione) is a nucleoside analog reverse transcriptase inhibitor-type antiretroviral drug that was approved for use against HIV/AIDS by the United States Food and Drug Administration (FDA) in 1987. Nevirapine (11-cyclopropyl-4-methyl-5,11-dihydro-6H-dipyrido[3,2-b:2′,3′-e][1,4]diazepin-6-one) is a non-nucleoside reverse transcriptase inhibitor-type antiretroviral drug that was approved for use against HIV/AIDS by the FDA in 1996. Since viral mutation and resistance may occur when patients are treated with either drug, the drugs are typically administered to adults as part of a drug cocktail of three or more anti-AIDS drugs.

Suitable excipients for the various embodiments disclosed herein include, for example, excipients which rapidly disintegrate in the mouth or aqueous solution, provide a compressible matrix that is suitable for granule formulation, and that provide a sweetening effect. Suitable excipients include, but is not limited to, LUDIFLASH® (commercially available from BASF, which comprises mannitol, poly[1-(2-oxo-1-pyrrolidinyl)-ethanediyl], polyvinylpyrrolidone-vinyl acetate copolymer, and sorbitol). Excipients may also include suspending agents, such as, but not limited to, microcrystalline cellulose/carboxymethyl cellulose sodium (such as, for example, AVICEL® RC-591 or AVICEL® CL-611, commercially available from FMC Biopolymer, Philadelphia, Pa.), hydroxypropyl methylcellulose, methylcellulose, hydroxypropyl cellulose, acacia, guar gum, and combinations of any thereof Suitable superdisintegrants may include, but are not limited to, croscarmellose sodium (sodium salt of a cross-linked, partly O-(carboxymethylated) cellulose), sodium starch glycolate, crosslinked polyvinylpyrrolidone (for example, KOLLIDON® CL-SF, commercially available from BASF, POLYPLASDONE® XL and POLYPLASDONE XL10, commercially available from ISP Technologies)), polyvinylpyrrolidone-vinyl acetate copolymer (for example, KOLLIDON® VA 64 FINE, commercially available from BASF), mucilage of plantago ovata, and combinations of any thereof Suitable sweeteners may include, but are not limited to, saccharin, mannitol, xylitol, sucralose, sucrose, aspartame, cyclamate, acesulfame potassium, and combinations of any thereof Suitable lubricants may include, for example, but not limited to, magnesium stearate, talc, fumed silica (for example, AEROSIL®, commercially available from Evonik Industries, Parsippany, N.J.).

According to certain embodiments, the pharmaceutical formulation for the reduction of MTCT of the virus may be in the form of a rapidly dissolving granule. According to these embodiments, the granules may be readily administered to a perinatal infant, such as an infant ranging in age from 0 (i.e., newborn) to 18 months in age, orally in a predetermined, age- or weight-appropriate dosage and interval. In the granule formulation, the pharmaceutical may be placed directly on the tongue of the infant where it can rapidly dissolve and be consumed by the patient. Alternatively, the granules may be sprinkled on or dissolved in a food product, for example, but not limited to, pudding or applesauce and then administered to the infant by feeding, for example by spoon. Further, the granules may be dissolved or suspended in an aqueous base liquid, such as water, juice, milk, or formula, and then administered to the infant. According to various embodiments the rapidly dissolving granule formulation may substantially dissolve in an aqueous liquid (such as water, milk, juice saliva, and the like) in less than 40 sec.

As discussed above, the various formulations for reducing MTCT of HIV in children includes a single active antiretroviral compound. According to one embodiment, the rapidly dissolving granule formulation for reducing MTCT of HIV may comprise a therapeutically effective amount of zidovudine as the active medicament in from 4% to 30% by weight of the total granule formulation, which in other embodiments may comprise from 4% to 10% by weight or even about 6% by weight of the total granule formulation. According to another embodiment, the rapidly dissolving granule formulation may comprise a therapeutically effective amount of nevirapine as the active medicament in from 2% to 25% by weight of the total granule formulation, which in other embodiments may comprise from 2% to 5% by weight or even about 3% by weight of the total granule formulation.

Further according to various embodiments, the non-active ingredients matrix of the granular formulation for reducing MTCT of HIV may include one or more excipients ranging from 50% to 95% of the total weight of the granule formulation, and in certain embodiments from 80% to 95%, or even 89.5% to 93% by weight of the total granule formulation. In specific embodiments, the one or more excipient may comprise LUDIFLASH® as a diluent-disintegration enhancer-sweetener excipient. The one or more superdisintegrant may comprise from 1% to 4% by weight of the granule formulation, and in certain embodiments, from 2% to 4% by weight. In specific embodiments, the superdisintegrant may comprise croscarmellose sodium in about 2% by weight and polyvinylpyrrolidone-vinyl acetate copolymer (such as KOLLIDON® VA 64 FINE) in about 2% by weight of the total granule formulation. As discussed in detail herein, the sweetener plays an important role in the palatability of the granule, since the bitterness of the antiretroviral drug can cause rejection of the treatment. Sweetener design must be carefully developed to effectively mask the taste of the drug. In specific embodiments, the sweetener may comprise from 0.1% to 5% by weight of the total granule formulation, and in certain embodiments from 0.1% to 1% by weight or even about 0.2% by weight of the formulation. In specific embodiments, the sweetener may be saccharin, for example, in an amount ranging from 0.1% to 1% by weight of the total granule or even about 0.2% by weight. In specific embodiments, the lubricants may comprise from 0.1% to 3% by weight of the total granule formulation, in other embodiments from 0.1% to 0.5% by weight or even about 0.25% by weight of the granule formulation. In specific embodiments, the lubricant may be fumed silica, such as, for example, AEROSIL®.

In another embodiment, the rapidly dissolving pharmaceutical formulation for reducing MTCT of HIV may be in the form of a rapidly dissolving tablet. Tablets may be suitable for infants ranging from 6 months to 18 months in age or older, who are able to readily swallow or chew the tablet. Tablets may provide further ease in dosing since they are of known dosages of the active medicament and can be readily scored and broken into smaller dosages. According to certain embodiments, the rapidly dissolving tablet formulation for reducing MTCT of HIV may comprise a therapeutically effective amount of zidovudine as the active medicament in from 5% to 30% by weight of the total tablet formulation, which in other embodiments may comprise from 15% to 25% by weight or even about 20% by weight of the total tablet formulation. According to another embodiment, the rapidly dissolving tablet formulation may comprise a therapeutically effective amount of nevirapine as the active medicament in from 5% to 25% by weight of the total tablet formulation, which in other embodiments may comprise from 15% to 25% by weight or even about 18% to 19% by weight of the total tablet formulation.

Further according to various embodiments, the non-active ingredients matrix of the rapidly dissolving tablet formulation for reducing MTCT of HIV may include excipients ranging from 15% to 75% of the total weight of the tablet formulation, and in certain embodiments from 60% to 75%, or even about 65% to 70% by weight of the total tablet formulation. In specific embodiments, the one or more excipient may comprise LUDIFLASH® as a diluent-disintegration enhancer-sweetener excipient. The one or more superdisintegrant may comprise from 1.5% to 8% by weight of the tablet formulation, and in certain embodiments, from 2% to 7% by weight, or even about 6% by weight of the total tablet formulation. For example, in specific embodiments, the superdisintegrant may comprise sodium starch glycolate, croscarmellose sodium or polyvinylpyrrolidone-vinyl acetate copolymer (such as KOLLIDON® VA 64 FINE) in either about 2% by weight or 6% by weight of the total tablet formulation. As discussed in detail herein, the one or more sweetener plays an important role in the palatability of the tablet, since the bitterness of the antiretroviral drug can cause rejection of the treatment. Sweetener design must be carefully developed to effectively mask the taste of the drug. In specific embodiments, the one or more sweetener may comprise from 2% to 11% by weight of the total tablet formulation, and in certain embodiments from 6% to 10% by weight. In specific embodiments, the one or more sweetener may comprise or be a combination of xylitol and sucralose, for example, xylitol in an amount ranging from 1.5% to 6% by weight of the total tablet or even about 5% by weight and sucralose in an amount ranging from 0.75% to 5% by weight of the total tablet or even about 3% to 3.5% by weight. In specific embodiments, the lubricants may comprise from 0.5% to 3% by weight of the total tablet formulation, in other embodiments from 0.5% to 1.5% by weight or even about 1% by weight of the tablet. In specific embodiments, the tablet may comprise magnesium stearate as a lubricant.

The rapidly dissolving granules for reducing MTCT of HIV may be administered to the patient in dosages according to age or weight of patient. For example, the granules may be provided in sachets or as a collection of granules (i.e., sprinkled on the tongue or on a liquid or viscous food product). According to various embodiments, the granules may have a mean particle size (geometric mean diameter) ranging from 50 microns to about 150 microns in diameter, or in other embodiments from about 100 microns or less. This allows for rapid dissolution of the granules on the tongue or in solution. The granules may be provided in sachets containing a premeasured dosage. For example, each sachet may contain from 100 mg to 300 mg of granules. The granules can be readily administered orally to neonates, perinatal infants or children below 3 years (for example, from 0 to 18 months in age), who may be too young to take tablets. In other embodiments, the rapidly dissolving tablet form may be used for children between 6 months and 18 months in age, or even up to 3 years in age, to prevent MTCT of HIV where the child is old enough to take a tablet form of the drug. According to these embodiments, the tablet may be of a known, fixed dosage, such as ranging from 200 mg to 400 mg in total tablet weight, or in other embodiments about 300 mg to 350 mg in total weight. One skilled in the art will recognize that other tablet sizes are within the scope of the present disclosure. Tablets may be made using a direct compression method and may be scored so that a fraction of the tablet having a known dosage can be administered to a child according to the age or weight of the child. The rapidly dissolving tablet, granular or powdered form may make it easier for children to take the medication without the need for water. This can be important in areas where potable water is scarce and may greatly improve compliance and/or adherence to a treatment regimen, which is critical to the use and efficacy of antiretroviral drugs and the prevention of MTCT of the virus. Further, since the HIV virus is known to develop resistance to specific therapies in cases of non-adherence to the treatment regimen, leading to treatment failure, the simplicity of use of the present treatments may improve adherence and therefore reduce development of drug resistance in the patient. Development of zidovudine or nevirapine into a single medicament, fast disintegrating, fast dissolving tablet or granules has been designated as an urgent priority by the World Health Organization (World Health Organization (WHO) report: WHO Expert Working Group Meeting to determine preferred ARV (antiretroviral) medicines for treating and preventing HIV infection in younger children, 2006).

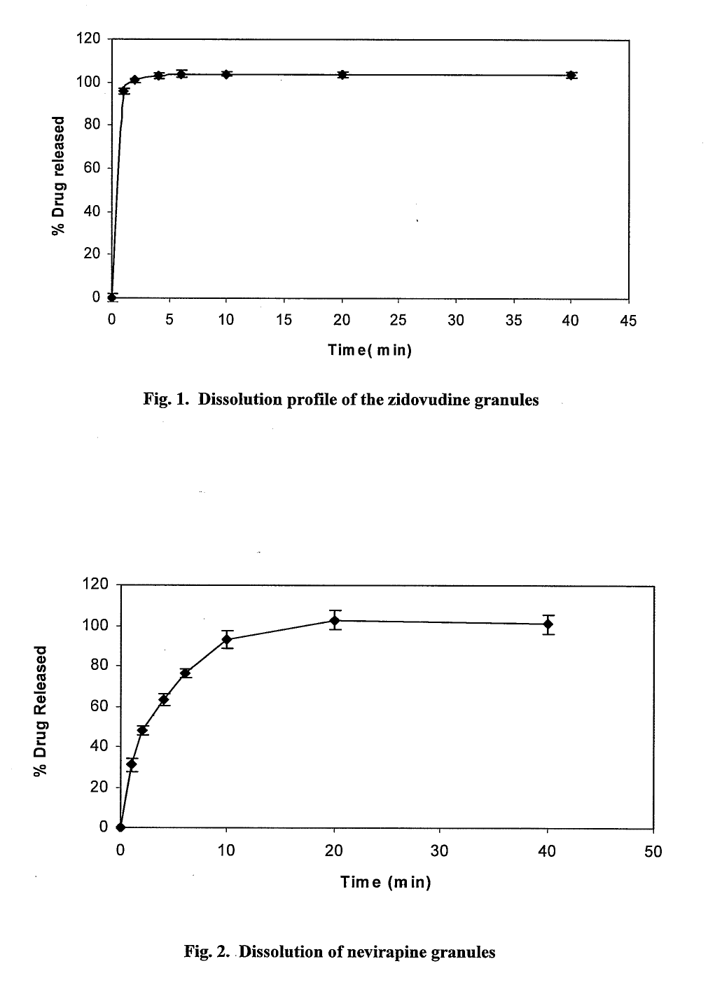

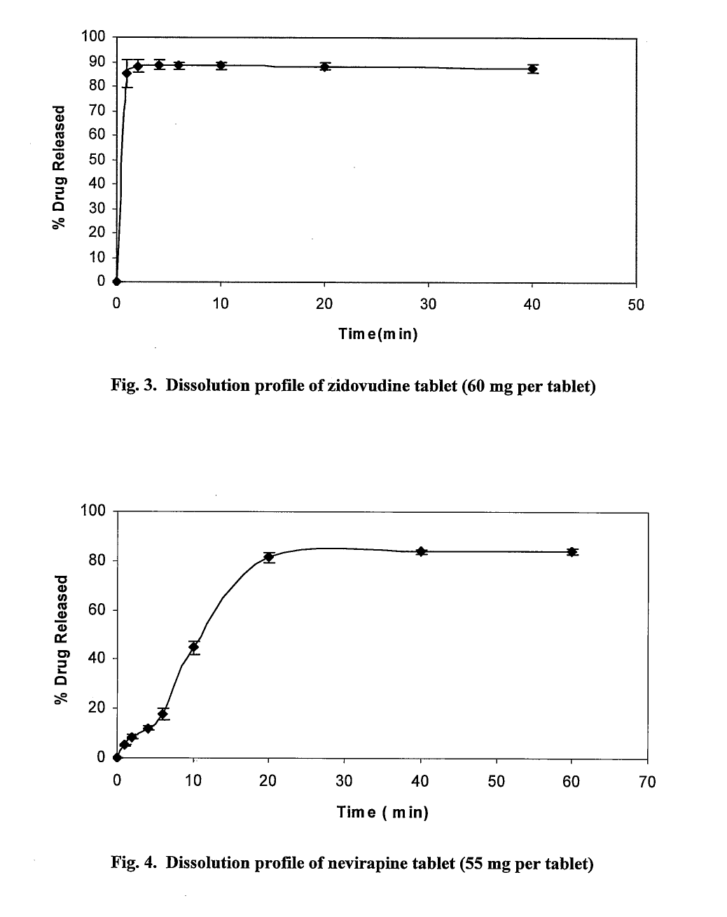

Both the rapidly dissolving granule formulations and rapidly dissolving tablet formulations for reducing the incidence of MTCT of HIV show acceptable stability and dissolutions profiles and release of the active medicaments in dissolution studies (using modified USP Type 1, see USP<711> in USP 30). FIG. 1 illustrates the dissolution profile of one embodiment of the zidovudine granule formulation demonstrating rapid dissolution of the formulation and availability of the medicament. Similarly, FIG. 2 illustrates the dissolution profile of one embodiment of the nevirapine granule formulation showing rapid dissolution of the formulation and availability of the medicament. FIG. 3 illustrates the dissolution profile of one embodiment of the zidovudine tablet formulation demonstrating rapid dissolution of the formulation and availability of the medicament. Likewise, FIG. 4 illustrates the dissolution profile of one embodiment of the nevirapine tablet formulation showing rapid dissolution of the formulation and availability of the medicament.

The taste of all formulations for reducing the incidence of MTCT of HIV were analyzed using a taste score that ranged between 0 and 4, where 0=bitter and 4=very sweet. According to these embodiments, the taste of the zidovudine granule formulation was 3.5 and the taste of the nevirapine granule formulation was 3.0. For the rapidly dissolving tablet formulations the taste was acceptable ranging from 3.0 to 3.5. The optimized taste of the formulations makes the pharmaceutical formulations more palatable to children.

Methods of reducing the incidence of or preventing MTCT of HIV are also within the scope of the present disclosure. For example, the present disclosure includes such methods which may comprise administering any of the pharmaceutical formulations described herein comprising zidovudine or nevirapine, either in granular or tablet form, to a neonatal, perinatal or child less than 3 years of age. The methods may include sprinkling the granules (or broken tablets) directly on the tongue of the patient or combining the granules or tablet with a food composition and feeding the mixture to the patient. The granules or tablets may also be mixed with an aqueous solution, such as, for example, water, milk, juice, formula, or other readily consumed liquid in which the formulation is substantially soluble or may be form a suspension and then administered to the patient. Alternatively, the methods may include administering a tablet form of the pharmaceutical formulations (either as a whole tablet, multiple tablets, or fraction of a tablet) directly to the patient. Also disclosed is the use of the pharmaceutical formulations according to any of the various embodiments described herein in the manufacture of a rapidly dissolving formulation for reducing or preventing the incidence or occurrence of mother to child transmission of HIV is also considered in the present disclosure. As described in detail herein, the pharmaceutical formulation may be used in either a rapidly dissolving granular formulation or a rapidly dissolving tablet formulation.

Still other embodiments of the present disclosure provide for a pharmaceutical treatment for HIV/AIDS in children. Treatments for HIV/AIDS in children described herein, differ from treatments for the prevention of MTCT of HIV, since treatments for HIV/AIDS are directed to young children (less than 16 years of age, for example, from 3 years to 16 years in age) who are infected with HIV and may also be demonstrating symptoms associated with AIDS. As discussed herein, prior treatment protocols for HIV/AIDS have typically focused on infected adults, in part, due to the large number of adults infected with HIV/AIDS relative to the number of children and certain issues associated with developing treatments for children. Typically, formulations have been developed based on adult models and studies utilizing adult patients. Given the disparity between the numbers of patients suffering from HIV/AIDS, formulations specifically developed for treating children have not been a large focus of the medical community. In general, children have typically been treated using formulations developed for adults using reduced dosage levels and prepared extemporaneously instead of using formulations specifically developed for children.

The pharmaceutical formulations for treating HIV/AIDS in children may take the form of rapidly dissolving reconstitutable granules comprising a combination of antiretroviral medications that may be reconstituted to form a liquid suspension or in other embodiments, may be in the form of a rapidly dissolving tablet with a fixed dose combination of antiretroviral medicaments. The development of a fixed dose drug combination of antiretroviral drugs into a fast disintegrating, fast dissolving granule or tablet formulation for treating children greater than 3 years old who are infected from HIV/AIDS has been designated as an urgent priority by the World Health Organization.

According to one embodiment, the present disclosure provides a rapidly dissolving pharmaceutical formulation for the treatment of HIV/AIDS in children. According to this embodiment, the pharmaceutical formulation may comprise lamivudine in from 0.4% to 10% by weight of the total formulation, zidovudine in from 0.65% to 25% by weight of the total formulation, and a non-active ingredients matrix. Lamivudine (2′,3′-dideoxy-3′-thiacytidine (“3TC”), 4-amino-1-[(2R,5S)-2-(hydroxymethyl)-1,3-oxathiolan-5-yl]-1,2-dihydropyrimidin-2-one) is a nucleoside analog reverse transcriptase inhibitor that was approved by the United States FDA in 1995 for use against HIV/AIDS. Thus, specific embodiments described herein may comprise two antiretroviral drugs (i.e., zidovudine and lamivudine), referred to herein as a 2 in 1 granule or tablet formulation. In particular embodiments, the various pharmaceutical formulations for treating HIV/AIDS in children may further comprise a third antiretroviral drug, nevirapine. Those embodiments of the rapidly dissolving pharmaceutical formulations comprising the third antiretroviral drug (i.e., nevirapine) are referred to herein as a 3 in 1 drug granule or tablet formulation. In specific embodiments, the nevirapine may comprise from 0.5% to 20% by weight of the total formulation. According to these embodiments, the 3 drug combination may provide extra protection against viral resistance to the two drug combination.

According to the various embodiments of the pharmaceutical formulation for treating HIV/AIDS in children, the non-active ingredients matrix may comprise one or more excipient comprising from 1.0% to 95% by weight of the total formulation, one or more superdisintegrant comprising from 0% (i.e., the formulation, such as a granule, contains no superdisintegrant) to 8% by weight of the total formulation, one or more sweeteners comprising from 0.75% to 92.52% by weight of the total formulation, one or more flavorants comprising from 0% to 0.20% by weight of the total formulation, one or more lubricants comprising from 0% to 3% by weight of the total formulation, and one or more parabens preservative comprising from 0% to 0.44% by weight of the total formulation. According to these embodiments, the pharmaceutical formulation may be in the form of a rapidly dissolving tablet or a rapidly dissolving granule, wherein the pharmaceutical formulation substantially dissolves or forms a suspension in an aqueous solution in less than 60 seconds.

According to certain embodiments, the rapidly dissolving pharmaceutical formulation for treating HIV/AIDS in children may be in a rapidly dissolving tablet formulation having a fixed dosage of the active medicaments. As described herein, a tablet formulation may provide specific benefits for administration of medicaments to children, such as the capability to tailoring the dosage to the child's age or weight, by scoring and breaking the tablet into fractions. According to these embodiments, the pharmaceutical formulation may be in the form of a rapidly dissolving tablet ranging in weight from about 200 mg to about 400 mg, or in other embodiments from about 300 mg to about 350 mg. The skilled artisan will recognize that other tablet sizes are possible and within the intended scope of the present disclosure.

According to those embodiments where the pharmaceutical formulation has a rapidly dissolving tablet form, the tablet may have the following composition. In certain embodiments, the zidovudine may comprise from 4.5% to 25% by weight of the total tablet formulation and in other embodiments from 15% to 20% by weight or even about 17% to 18% by weight of the total tablet formulation. The lamivudine may comprise from 2.2% to 10% by weight of the total tablet formulation and in other embodiments from 5% to 10% by weight or even from 7% to 8% by weight of the total tablet. The one or more excipients may comprise from 12% to 55% by weight of the total tablet formulation and in certain embodiments from 35% to 55% by weight or even from about 45% to 46% by weight of the total formulation. Suitable excipients may include LUDIFLASH® as a diluent-disintegration enhancer-sweetener excipient. The one or more superdisintegrants, such as, for example, croscarmellose sodium, sodium starch glycolate, crosslinked polyvinylpyrrolidone, or polyvinylpyrrolidone-vinyl acetate copolymer, may comprise from 1.5% to 8% by weight of the total tablet formulation and in certain embodiments from 5% to 7% by weight or even about 6% by weight of the total formulation. The one or more sweeteners (for example, mannitol, saccharin, sucralose, xylitol, sucrose, aspartame, acesulfame potassium, and cyclamate) may comprise from 0.75% to 12% by weight and in certain embodiments from 2% to 11% by weight of the total formulation. In specific embodiments, the one or more sweeteners may comprise xylitol in from 1.5% to 6% by weight and/or sucralose in from 0.75% to 6% by weight of the total formulation. In one specific embodiment, the one or more sweetener may comprise about 5% xylitol and about 5% of sucralose by weight of the total formulation. Specific embodiments of the tablet may comprise one or more flavorants may be one or more natural or artificial flavorant (such as, but not limited to cherry, grape, raspberry, bubble gum, or mixed fruit flavorant) comprising from 0.0001% to 0.20% by weight of the total tablet formulation. The one or more lubricants, such as magnesium stearate, talc or fumed silica, may comprise from 0.5% to 3% by weight of the total tablet formulation or even about 1% by weight of the total formulation. In specific embodiments, the tablet formulation comprises magnesium stearate.

In those embodiments where the rapidly dissolving tablet formulation comprises zidovudine, lamivudine, and nevirapine (i.e., the 3 in 1 tablet formulation), the tablet may comprise nevirapine in from 4% to 20% by weight of the tablet and in certain embodiments from 10% to 20% by weight or even 15% to 16% by weight of the total tablet formulation.

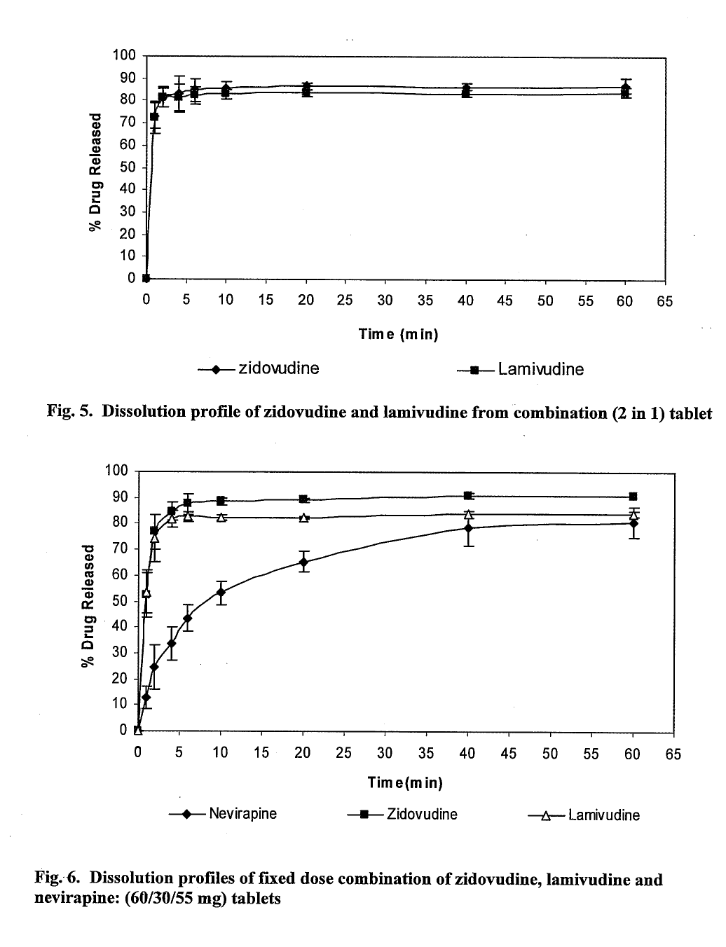

Both the 2 in 1 tablet formulation and the 3 in 1 tablet formulation displayed satisfactory dissolution and release of the active medicaments in dissolution studies. For the 2 in 1 tablet, more than 80% of the zidovudine and lamivudine were released within 5 minutes. FIG. 5 illustrates the dissolution profile of one embodiment of the 2 in 1 tablet formulation demonstrating rapid dissolution of the formulation and availability of the medicaments. Similarly, in the 3 in 1 tablet, more than 80% of the zidovudine and lamivudine were released within 6 minutes and the nevirapine was released within 60 minutes. FIG. 6 illustrates the dissolution profile of one embodiment of the 3 in 1 tablet formulation showing rapid dissolution of the formulation and availability of the medicaments.

According to certain embodiments, the rapidly dissolving pharmaceutical formulation for the treatment of HIV/AIDS in children may be in a reconstitutable granular formulation having a fixed dosage of the active medicaments. As described herein, a reconstitutable granular formulation may provide specific benefits for administration of medicaments to children, such as the availability of tailoring the dosage to the child's age or weight, by reconstituting specific amounts of the granules in solution. The granule formulation may demonstrate desired properties such as taste masking, lack of drug-excipient interaction, and acceptable flow characteristics. The reconstitutable type granule formulation may be reconstituted with water or other aqueous solution to form a stable suspension. Because the active medicaments are in a liquid suspension, this allows for a flexible dosing, as the volume of the liquid suspension that is orally administered can be varied according to the preferred dosage according to age or weight of the child. The reconstitutable suspension form has distinct advantages since the liquid state may be administered orally and is easier for children or even older patients who have difficulty swallowing to take. Further, the reconstitutable granule formulation has a shelf-life at room temperature of approximately three years (determined from the shelf life of samples stored at 40° C.) and the reconstituted suspension will have a stability of up to one month with or without refrigeration. This allows for the treatment to be utilized in economically depressed areas (such as undeveloped or third world countries, for example, in Asia or sub-Saharan Africa) where refrigeration may not be readily available and eases use of the treatment protocol in such areas, thereby increasing adherence to the protocol.

According to these embodiments, the pharmaceutical formulation may be in the form of rapidly dissolving reconstitutable granules having a particle size of greater than 35 mesh, or in other embodiments, having a geometric mean diameter for particle size ranging from about 200 microns to 400 microns, and in certain embodiments ranging from 250 microns to 350 microns or even from about 300 microns to 325 microns in diameter. The skilled artisan will recognize that other granule sizes are possible and within the intended scope of the present disclosure.

According to those embodiments where the pharmaceutical formulation has a rapidly dissolving reconstitutable granule form, the granule may have the following composition. In certain embodiments, the zidovudine may comprise from 0.65% to 6.5% by weight of the total granule formulation. In one embodiment where the pharmaceutical formulation is a 2 in 1 granule formulation, the zidovudine may comprise from 1.0% to 6.5% by weight or even about 5.45% by weight of the total granule formulation. In another embodiment where the pharmaceutical formulation is a 3 in 1 granule formulation, the zidovudine may comprise from 0.65% to 3.3% by weight or even about 2.66% by weight of the total granule formulation. The lamivudine may comprise from 0.4% to 2.65% by weight of the total granule formulation. In one embodiment where the pharmaceutical formulation is a 2 in 1 granule formulation, the lamivudine may comprise from 0.4% to 2.65% by weight or even about 2.18% by weight of the total granule formulation. In another embodiment where the pharmaceutical formulation is a 3 in 1 granule formulation, the lamivudine may comprise from 0.44% to 1.77% by weight or even about 1.33% by weight of the total granule formulation. The one or more excipients may comprise a suspending agent from 1.0% to 5.4% by weight of the total granule formulation and in certain embodiments from 3.0% to 5.0% by weight or even about 4.4% by weight of the total formulation. Suitable suspending agents may include microcrystalline cellulose and carboxymethyl cellulose sodium, for example, AVICEL® RC 591, a mixture of microcrystalline cellulose and carboxymethylcellulose sodium, commercially available from FMC Biopolymers, or any of the other suspending agents described herein. The suspending agent may function first as a binder during the granulation process and then may function as a suspending agent when the granule formulation is mixed with water or other aqueous solution. The one or more sweeteners may comprise from 25% to 92.52% by weight and in certain embodiments about 87.6% to about 89.0% by weight of the total granule formulation. In specific embodiments, the one or more sweeteners may comprise xylitol in from 25% to 92% by weight and/or sucralose in from 0.08% to 0.52% by weight of the total granule formulation. In one specific embodiment, the one or more sweetener may comprise about 87% to about 89% xylitol by weight and about 0.44% sucralose by weight of the total granule formulation. The one or more parabens preservative may comprise from 0.008% to 0.44% by weight of the granule formulation. In one specific embodiment, the parabens preservative may comprise methyl parabens in from about 0.075% to 0.40% by weight of the granule formulation and propyl parabens in from about 0.008% to 0.038% by weight of the granule formulation, and in certain embodiments the parabens may comprise methyl parabens in about 0.33% by weight and propyl parabens in about 0.03% by weight of the granule formulation. The one or more flavorant (such as natural or artificial cherry, grape, raspberry, bubble gum or mixed fruit flavorant) may comprise from 0.0001% to 0.20% by weight of the granule formulation or even about 0.0004% by weight of the granule formulation. According to certain embodiments, the granule formulation may also comprise a superdisintegrant and/or a lubricant, as described herein. For example, embodiments of a rapidly dissolving granule formulation may include a superdisintegrant and/or a lubricant, whereas embodiments of the reconstitutable granule may include a lubricant.

In those embodiments where the reconstitutable granule formulation comprises zidovudine, lamivudine, and nevirapine (i.e., a 3 in 1 granule formulation), the granule may further comprise nevirapine in from 0.5% to 2.7% by weight of the total tablet formulation and in certain embodiments from 1.5% to 2.5% by weight or even about 2.2% by weight of the total tablet formulation.

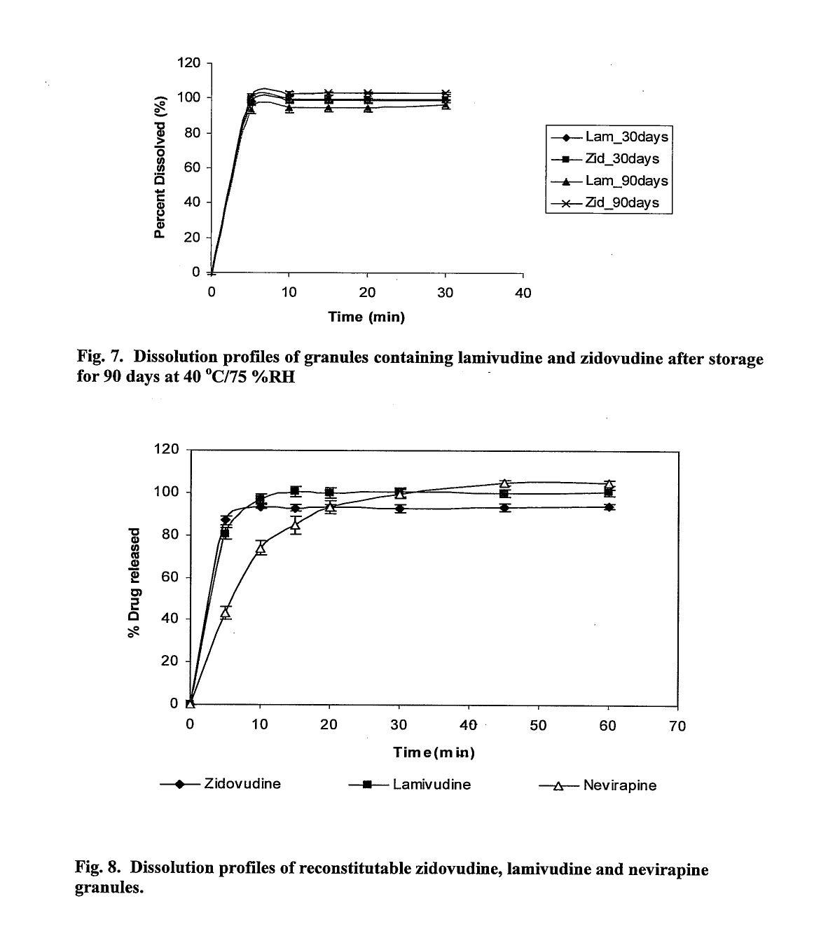

Both the 2 in 1 reconstitutable granule formulation and the 3 in 1 reconstitutable granule formulation displayed satisfactory dissolution and release of the medicaments in dissolution studies. For the 2 in 1 granule, more than 80% of the zidovudine and lamivudine were released within 5 minutes. FIG. 7 illustrates the dissolution profile of one embodiment of the 2 in 1 granule formulation demonstrating rapid dissolution of the formulation and availability of the medicaments. FIG. 8 illustrates the dissolution profile of one embodiment of the 3 in 1 granule formulation showing rapid dissolution of the formulation and availability of the medicaments.

The 2 in 1 or 3 in 1 reconstitutable granules according to the various embodiments of the pharmaceutical formulations may be reconstituted in water or other aqueous solution to form a stable, liquid suspension that may be orally administered to a patient (i.e., a child infected with HIV/AIDS) at predetermined dosages and intervals. In certain embodiments, the granule formulation may be reconstituted in water or other aqueous solution in a percentage range of from 30% weight of the granule per volume of liquid (% w/v) to 46% w/v, and in other embodiments in about 45% w/v. Suspensions having these concentrations (% w/v) correspond to an effective dose of from 4.5 mg/mL to 12 mg/mL of lamivudine, from 9 mg/mL to 30 mg/mL of zidovudine, and, in those embodiments which also include nevirapine, the nevirapine will have an effective dose of from 7.5 mg/mL to 10 mg/mL. The actual effective dose needed will vary based on the weight or age or the child treated, however, because the active agents are in a liquid suspension, the effective dose administered may be readily tailored by varying the volume of the liquid suspension (and consequently the amount of active medicament) administered to the child. The pH of the formulation ranges from between about 6.20 to about 6.70 and in specific embodiments the pH may be about 6.4.

For reconstituted suspensions, the granule may have a weight per volume composition as follows. The zidovudine may comprise from 0.4% to 3.6% w/v of the total liquid formulation and in other embodiments from 1.0% to 2.0% w/v or even about 1.2% w/v of the total liquid formulation. The lamivudine may comprise from 0.2% to 1.2% of the total liquid formulation and in other embodiments from 0.3% to 0.8% w/v or even about 0.6% w/v of the total liquid formulation. The one or more excipients may comprise a suspending agent in from 0.5% to 4.0% w/v of the total liquid formulation and in certain embodiments from 1.0% to 3.0% w/v or even about 2.0% w/v of the total liquid formulation. The one or more sweeteners may comprise from 15% to 50.5% w/v and in certain embodiments about 40.2% w/v of the total liquid formulation. In specific embodiments, the one or more sweeteners may comprise xylitol in from 15% to 50% w/v and/or sucralose in from 0.05% to 0.5% w/v of the total liquid formulation. In one specific embodiment, the one or more sweetener may comprise about 40% w/v xylitol and about 0.2% w/v sucralose in the total liquid formulation. The one or more parabens preservative may comprise from 0.005% to 0.22% w/v of the formulation. In one specific embodiment, the parabens preservative may comprise methyl parabens in from about 0.05% to 0.2% w/v of the liquid formulation and propyl parabens in from about 0.005% to 0.02% w/v of the liquid formulation, and in certain embodiments the parabens may comprise methyl parabens in about 0.15% w/v and propyl parabens in about 0.015% w/v of the liquid formulation. In certain embodiments the granules may also comprise a flavorant (such as natural or artificial cherry, grape, raspberry, bubble gum, or mixed fruit flavorant) in from 0.0001% to 0.20% w/v of the formulation.

The granule formulations according to the various embodiments described herein are physically stable for at least 3 months according to International Conference on Harmonization (ICH Q1AR2) recommended storage conditions of 40° C./75% relative humidity (RH). Properties such as flowability, particle size distribution, content uniformity, and dissolution either remained unchanged or minimally changed within acceptable criteria limits during the storage time. Because the various formulations for treating HIV/AIDS described in detail herein are intended for children, the taste is very important. The taste of the 2 in 1 and 3 in 1 reconstitutable granule formulations was analyzed using the non-parametric Chi-square distribution with a p-value of >0.05 indicating non-significance. According to these studies, the Chi-square test of taste of xylitol displayed a p-value of 0.0001, indicating a significant effect. Thus, in certain embodiments of the various formulations of the 2 in 1 or 3 in 1 formulations high concentrations of xylitol may be important for palatability of the formulations.

Chemical potency of the 2 in 1 or 3 in 1 reconstitutable granule formulations was not less than 100%. The reconstituted suspensions were stable for at least one month (ICH Q1AR2) conditions of 30° C./65% RH in terms of taste, viscosity, and potency. The percent potency of the suspension was at least 98.66% after storage and acceptable antimicrobial properties of the reconstituted suspensions were maintained for at least 28 days, the within-use period after reconstitution.

Methods of reducing the treating HIV/AIDS are also within the scope of the present disclosure. For example, the present disclosure includes such methods which may comprise administering any of the pharmaceutical formulations for treating HIV/AIDS described herein comprising zidovudine and lamivudine (2 in 1 formulations), and in certain embodiments, nevirapine (3 in 1 formulations), either in tablet form or as a reconstituted solution from the granule form, to child from 3 years to 16 years of age who is infected with HIV or exhibiting symptoms of AIDS. When the formulation is a rapidly dissolving tablet formulation, the tablet may be orally administered directly to the child, or when necessary scored and broken into fractions depending on the age or weight of the child. In those embodiments where the pharmaceutical is a reconstitutable granule, the method may comprise reconstituting the granules in an aqueous liquid to form a stable suspension and orally administering at least a portion of the suspension to the child. The volume or amount of the suspension that is orally administered may be determined based on the age and/or weight of the patient. The use of the pharmaceutical formulation according to any of the various embodiments described herein in the manufacture of a rapidly dissolving formulation for the treatment of HIV or AIDS in children is also considered in the present disclosure. As described in detail herein, the rapidly dissolving pharmaceutical formulation may be used in either a tablet formulation, a rapidly dissolving granule formulation, or a reconstitutable granule formulation, as described herein.

While various specific embodiments have been described in detail herein, the present disclosure is intended to cover various different combinations of the disclosed embodiments and is not limited to those specific embodiments described herein. Various embodiments of the present disclosure will be better understood when read in conjunction with the following non-limiting Examples. The procedures set forth in the Examples below are not intended to be limiting herein, as those skilled in the art will appreciate that various modifications to the procedures set forth in the Examples, as well as to other procedures not described in the Examples, may be useful in practicing the invention as described herein and set forth in the appended claims.

### EXAMPLES

### Example 1

### Rapidly Dissolving Granules for Reducing Incidence of MTCT of HIV

The rapidly dissolving granule formulations for reducing the incidence of MTCT of HIV including the active medicament zidovudine or nevirapine were made using the following protocol. In vitro dissolution tests were carried out for the granules to monitor the drug release profile.

The composition of one embodiment of the zidovudine granule formulation is presented in Table 1 and the composition of one embodiment of the nevirapine granule formulation is presented in Table 2. The granules are made by mixing a sweetener (saccharin, 0.20% w/w) and a superdisintegrants (croscarmellose sodium and KOLLIDON® VA 63 FINE, 2% w/w) each in a low shear planetary mixer. This is followed with the addition of the active medicament 6.0% w/w zidovudine or 3.0% w/w nevirapine) and the excipient (LUDIFLASH®). Wet granulation is done by the addition of 30% water (which can be reduced upon increasing batch size). Drying of the wet granules is done at 25° C. followed by screening through a QUADRO® COMIL® screen mill. The lubricant is then blended with the granules to form the final granule product. The formulation is developed such that 200 mg of granules contains 12 mg or 6% w/w zidovudine or 6 mg or 3.0% w/w nevirapine.

TABLE 1
Fast Disintegrating Zidovudine Granules

| | mg/sachet | % w/w |
|---|---|---|
| Zidovudine | 12.0 | 6.0 |
| LUDIFLASH ® | 179.1 | 89.55 |
| Saccharin | 0.4 | 0.20 |
| Croscarmellose sodium | 4.0 | 2.00 |
| KOLLIDON ® - VA 64 Fine | 4.0 | 2.00 |
| AEROSIL ® | 0.5 | 0.25 |
| Total | 200.0 | 100.00 |

TABLE 2
Fast Disintegrating Nevirapine Granules

| | mg/sachet | % w/w |
|---|---|---|
| Nevirapine | 6.0 | 3.0 |
| LUDIFLASH ® | 185.1 | 92.55 |
| Saccharin | 0.4 | 0.20 |
| Croscarmellose sodium | 4.0 | 2.00 |
| KOLLIDON ® - VA 64 Fine | 4.0 | 2.00 |
| AEROSIL ® | 0.5 | 0.25 |
| Total | 200.0 | 100.00 |

The taste of all the formulations was determined using a taste score that ranged between 0 and 4, with a taste score of 0 being bitter and 4 being very sweet. The taste for the zidovudine granule formulation was 3.5 while that of nevirapine granule formulation was 3.0.

The zidovudine or nevirapine granules wetted within 30 and 40 sec. respectively. The drug release study was carried out using USP Type 1 method. A dissolution medium of 900 mL of deionized water was used for zidovudine. Similarly, a phosphate buffer of pH 2.0±0.02 was used for nevirapine as a dissolution medium. Samples (n=6) representing one dose each were accurately weighed and transferred in six baskets, respectively. The baskets were set at 100 RPM and the dissolution medium was maintained at 37° C.±0.2° C. Samples (2 mL) were withdrawn at the intervals of 1, 2, 4, 6, 10, 20, 40, and 60 minutes. The dissolution profiles for zidovudine and nevirapine are shown in FIGS. 1 and 2, respectively.

### Example 2

### Rapidly Dissolving Tablets for Reducing Incidence of MTCT of HIV

The rapidly dissolving tablet formulations for reducing the incidence of MTCT of HIV including the active medicament zidovudine or nevirapine were made using the following protocol. The tablet formulations were made using the direct compression methods. The tablets are scored so that they can be broken into fractions containing age or weight appropriate doses of the medicament. In vitro dissolution tests were carried out for the tablets to monitor the drug release profile.

For the zidovudine tablet formulation, a 3×2 factorial design (3 superdisintegrants each at two levels) was implemented (Table 3) to see the effect of the superdisintegrant on reducing the disintegration time of the targeted zidovudine single dose (60 mg) tablet. The active component was blended with the excipients (LUDIFLASH®), the superdisintegrants (sodium starch glycolate (SSG), croscarmellose sodium (CC) or crosslinked polyvinylpyrrolidone (KOLLIDON® CL-SF)) at the two different amounts, the sweeteners xylitol (XYLITAB® 100, commercially available from Danisco A/S, Denmark) and sucralose, and the lubricant (magnesium stearate) were mixed for a suitable amount of time. Tablets (targeted weight ˜300 mg) were compressed in the Carver Press using an 11 mm die and punch set at a compression of 11.12 KN (˜2500 lbs). The compressed tablets were stored in an air-tight container until further physical tests were performed.

TABLE 3
Zidovudine (60 mg Tablet) - Single Dose

| | | Amt per | % w/w | Amt per | % w/w |
|---|---|---|---|---|---|
| | | tablet | per | tablet | per |
| Ingredients: | Category | (mg) | tablet | (mg) | tablet |
| Zidovudine | Active | 60 | 20.00 | 60 | 20.00 |
| LUDIFLASH ® | Diluent/Disintegrant/ | 205.5 | 68.50 | 193.5 | 64.50 |
| | sweetening agent | | | | |
| SSG, CC or KOLLIDON ® | Disintegrant | 6 | 2.00 | 18 | 6.00 |
| XYLITAB ® 100 | Sweetener | 15 | 5.00 | 15 | 5.00 |
| Sucralose | Sweetener | 10.5 | 3.50 | 10.5 | 3.50 |
| Magnesium Stearate | Lubricant | 3 | 1.00 | 3 | 1.00 |
| Total tablet weight | | 300 | 100.00 | 300 | 100.00 |

A similar formulation and testing protocol for the nevirapine tablet formulation, (i.e., a 3×2 factorial design (3 superdisintegrants each at two levels)) was implemented (Table 4) to see the effect of the superdisintegrant on reducing the disintegration time of the targeted nevirapine single dose (55 mg) tablet. The active component was blended with the excipients (LUDIFLASH®), the superdisintegrants (sodium starch glycolate (SSG), croscarmellose sodium (CC) or crosslinked polyvinylpyrrolidone (KOLLIDON® CL-SF)) at the two different amounts, the sweeteners xylitol (XYLITAB® 100) and sucralose, and the lubricant (magnesium stearate) were mixed for a suitable amount of time. Tablets (targeted weight ˜300 mg) were compressed in the Carver Press using an 11 mm die and punch set at a compression of 11.12 KN (˜2500 lbs). The compressed tablets were stored in an air-tight container until further physical tests were performed.

TABLE 4
Nevirapine (55 mg Tablet) - Single Dose

| | | Amount | | Amount | |
|---|---|---|---|---|---|
| | | per | % per | per | % per |
| | | tablet | tablet | tablet | tablet |
| Ingredients: | Category | (mg) | (w/w) | (mg) | (w/w) |
| Nevirapine | Active | 55 | 18.33 | 55 | 18.33 |
| LUDIFLASH ® | Diluent/Disintegrant/ | 212.00 | 70.70 | 200.00 | 6670 |
| | sweetening agent | | | | |
| SSG, CC or KOLLIDON ® | Super disintegrant | 6 | 2.00 | 18 | 6.00 |
| XYLITAB ® 100 | Sweetener | 15 | 5.00 | 15 | 5.00 |
| Sucralose | Sweetener | 9 | 3 | 9 | 3 |
| Magnesium Stearate | Lubricant | 3 | 1.00 | 3 | 1.00 |
| Total tablet weight | | 300 | 100.00 | 300 | 100.00 |

### Crushing Strength, Friability and Disintegration of Tablets:

The different physical parameters were determined using 5 tablets for each of the formulations of the zidovudine and nevirapine tablets. Tablet crushing strength was determined using the Electronik Tablet Hardness tester. Similarly, friability was determined using Roche Friability Tester which was set for 4 minutes at 25 RPM. The disintegration test was performed in 900 mL of simulated salivary fluid maintained at a temperature of 37° C.±0.2° C.

### Dissolution Studies:

The dissolution study was performed on the tablet formulations with lowest disintegration time with appreciable tablet crushing strength and friability. USP Type 1 method was used, with 900 mL of deionized water as the dissolution medium as stated in USP for zidovudine. For the nevirapine tablets, USP Type 1 method was used with 900 mL of phosphate buffer, pH 2.0±0.02, as the dissolution medium. The basket was set at 100 RPM and the dissolution medium was maintained at 37° C.±0.2° C. Samples were withdrawn at the intervals of 1, 2, 4, 6, 10, 20, 40 and 60 minutes respectively.

### Physical Parameters of Tablets for Reducing Incidence of MTCT of HIV

### Disintegration Test, Crushing Strength and Friability:

Zidovudine formulation with 2% of croscarmellose sodium has the lowest disintegration time of 17-31 sec. with appreciable crushing strength around 93 N and friability less than 1%. This formulation was chosen for the dissolution study (Table 5). Similarly, nevirapine tablets with 2% croscarmellose sodium were chosen which has the lowest disintegration time of 15-18 sec., a crushing strength of around 70 N and friability less than 1% (Table 6). All these values fall within the acceptable criteria. The taste of the tablets was acceptable ranging from 3.0-3.5 on the score scale referred to earlier.

TABLE 5
Physical Parameters for Zidovudine Tablets

| | Level | Average ( n = 5) | | |
|---|---|---|---|---|
| Superdisintegrant | (% w/w) | DT (sec) | CS (N) | F (%) |
| SSG | 2 | 28.8 ± 2.1 | 82 ± 7.8 | 0.55 |
| SSG | 6 | 27.3 ± 4..2 | 81.2 ± 4.1 | 0.41 |
| CC | 2 | 17.8 ± 1.9 | 93.6 ± 4.4 | 0.13 |
| CC | 6 | 21.2 ± 3.0 | 85.6 ± 4.5 | 0.39 |
| K-CLSF | 2 | 45.7 ± 4.8 | 82.8 ± 7.2 | 0.68 |
| K-CLSF | 6 | 22.8 ± 1.9 | 90.6 ± 7.4 | 0.54 |

TABLE 6
Physical parameters for Nevirapine Tablets

| | Level | Average ( n = 5) | | |
|---|---|---|---|---|
| Superdisintegrant | (% w/w) | DT (sec) | CS (N) | F (%) |
| SSG | 2 | 24.8 ± 1.7 | 65 ± 8.1 | 0.8 |
| SSG | 6 | 34.7 ± 3.2 | 62.2 ± 7.3 | 1.07 |
| CC | 2 | 15.7 ± 2.3 | 77.2 ± 10.8 | 0.8 |
| CC | 6 | 18.8 ± 3.0 | 64.4 ± 10 | 0.73 |
| K-CLSF | 2 | 21.2 ± 1.8 | 72.8 ± 8.8 | 0.47 |
| K-CLSF | 6 | 21.3 ± 1.5 | 87.5 ± 6.6 | 0.27 |

SSG: Sodium Starch Glycolate; CC: Croscarmellose sodium, K-CLSF: KOLLEDON ® CL-SF DT: Disintegration Time; CS: Crushing Strength; F: Friability (% loss in weight)

### Release of Medicament

For the rapidly dissolving zidovudine tablet formulation, more than 80% of the zidovudine was released within 30 minutes in compliance with the USP requirements that states that for immediate release dosage form, 80% of the labeled amount (60 mg in this case) is dissolved in 30 minutes (FIG. 3). For the rapidly dissolving nevirapine, more than 80% of the nevirapine was released within 60 minutes which complies with the USP requirements that states that for an immediate release dosage form, 75% of the labeled amount (55 mg in this case) is dissolved in 60 minutes (FIG. 4).

### Example 3

### Rapidly Dissolving Multi-Drug Tablets for Treating HIV/AIDS in Children

The rapidly dissolving tablet formulations for treating HIV/AIDS including a multi-drug combination of active medicaments were made using the following protocol. The 2 in 1 tablet formulation was prepared using effective amounts of zidovudine and lamivudine. The 3 in 1 tablet formulation was prepared using effective amounts of lamivudine, zidovudine, and nevirapine. The tablet formulations were made using the direct compression methods. The tablets are scored so that they can be broken into fractions containing age or weight appropriate doses of the medicament. In-vitro dissolution tests were carried out for the tablets to monitor the drug release profile.

### 2 in 1 Tablet Formulation

For the 2 in 1 tablet formulation containing zidovudine and lamivudine, a 3×3 factorial design (3 superdisintegrants each at three levels) was implemented (Table 7) to examine the effect of different superdisintegrants and superdisintegrants at various levels on reducing the disintegration time of the targeted fixed dose combination tablet: zidovudine/lamivudine (60/30 mg) tablet. The active components were blended with the excipients (LUDIFLASH®), the superdisintegrants (sodium starch glycolate (SSG), croscarmellose sodium (CC) or crosslinked polyvinylpyrrolidone (KOLLIDON® CL-SF)) at the three different amounts, the sweeteners xylitol (XYLITAB® 100) and sucralose, and the lubricant (magnesium stearate) were mixed for a suitable amount of time. Tablets (targeted weight ˜300 mg) were compressed in the Carver Press using an 11 mm die and punch set at a compression of 11.12 KN (˜2500 lbs). The compressed tablets were stored in an air-tight container until the physical tests were performed.

### Crushing Strength, Friability and Disintegration of 2 in 1 Tablets:

The different physical parameters were determined for various formulations of the 2 in 1 zidovudine/lamivudine tablets. Tablet crushing strength was determined using the Electronik Tablet Hardness tester. Similarly, friability was determined using Roche Friability Tester which was set for 4 minutes at 25 RPM. The disintegration test was performed in 900 mL of simulated salivary fluid maintained at a temperature of 37° C.±0.2° C.

### Dissolution Studies:

The dissolution study was performed on the formulation with lowest disintegration time with appreciable tablet crushing strength and friability. USP Type 1 method was used, with 900 mL of deionized water as the dissolution medium. The basket was set at 100 RPM and the dissolution medium was maintained at 37° C. ±0.2° C. Samples were withdrawn at the intervals of 1, 2, 4, 6, 10, 20, 40 and 60 minutes.

### Physical Parameters of 2 in 1 Tablets for Treating HIV/AIDS in Children

### Disintegration Test, Crushing Strength and Friability:

For the 2-in-1, fixed dose combination tablets addition of KOLLIDON® CL-SF in at least 2% level to the formulation exhibited a suitable disintegration time (Table 8). From the observed data, it is apparent that for fixed dose combination, addition of KOLLIDON® CL-SF at 6% level to the formulation exhibited a suitable disintegration time compared to at other levels. The crushing strength was around 74 N. This formulation was selected for further dissolution study. In the dissolution study, more than 80% of the drug for zidovudine and lamivudine were released within 5 minutes. This may be attributed to the highly soluble nature of lamivudine and zidovudine (FIG. 5).

TABLE 7
2 in 1 Combination Tablet (Zidovudine/Lamivudine
60/30 mg Tablet) Formulation

| | | Amt | % w/w | Amt | % w/w | Amt | % w/w |
|---|---|---|---|---|---|---|---|
| | | per | per | per | per | per | per |
| Ingredients: | Category | tablet | tablet | tablet | tablet | tablet | tablet |
| Zidovudine | Active | 60 | 20.00 | 60 | 20.00 | 60 | 20.00 |
| Lamivudine | Active | 30 | 10.00 | 30 | 10.00 | 30 | 10.00 |
| LUDIFLASH ® | Diluent/ | 171 | 57 | 171 | 57 | 171 | 57 |
| | Disintegrant/ | | | | | | |
| | sweetening | | | | | | |
| | agent | | | | | | |
| KOLLIDON CL-SF, | Super | 6 | 2 | 12 | 4 | 18 | 6 |
| Croscarmellose sodium or | disintegrant | | | | | | |
| Sodium starch glycolate | | | | | | | |
| Sucralose | Sweetener | 15 | 5 | 15 | 5 | 15 | 5 |
| Xylitol | Sweetener | 15 | 5.00 | 15 | 5.00 | 15 | 5.00 |
| Magnesium Stearate | Lubricant | 3 | 1.00 | 3 | 1.00 | 3 | 1.00 |
| Total tablet weight | | 300 | 100.0 | 306 | 102.0 | 312 | 104.0 |

TABLE 8
Physical Parameters for 2 in 1 Combination Tablets

| | Level | Average (n = 5) | | |
|---|---|---|---|---|
| Superdisintegrant | (% w/w) | DT (sec) | CS (N) | F (%) |
| SSG | 2 | 75.3 ± 9.0 | 72.8 ± 7.2 | 0.8 |
| SSG | 4 | 49 ± 11.9 | 66 ± 10.8 | 0.39 |
| SSG | 6 | 47.7 ± 8.1 | 66.6 ± 9.6 | 0.83 |
| CC | 2 | 36.7 ± 5.4 | 77.6 ± 10.6 | 0.27 |
| CC | 4 | 42.5 ± 4.2 | 79.2 ± 7.5 | 0.46 |
| CC | 6 | 39.8 ± 2.3 | 75.8 ± 7.3 | 0.2 |
| K-CLSF | 2 | 28.3 ± 6.7 | 82.0 ± 8.9 | 0.53 |
| K-CLSF | 4 | 26.3 ± 5.5 | 66.4 ± 5.3 | 0.33 |
| K-CLSF | 6 | 27.7 ± 0.8 | 74.4 ± 6.1 | 0.32 |

SSG: Sodium Starch Glycolate; CC: Croscarmellose sodium, K-CLSF: KOLLIDON ® CL-SF DT: Disintegration Time; CS: Crushing Strength; F: Friability (% loss in weight)

### 3 in 1 Tablet Formulation

For the 3 in 1 tablet formulation containing zidovudine, lamivudine, and nevirapine, a 3×3 factorial design (3 superdisintegrants each at three levels) was implemented (Table 9) to examine the effect of different superdisintegrants and superdisintegrant level on reducing the disintegration time of the fixed dose combination tablet: zidovudine/lamivudine/nevirapine (60/30/55 mg) tablet. The active components were blended with the excipients (LUDIFLASH®), the superdisintegrants (sodium starch glycolate (SSG), croscarmellose sodium (CC) or crosslinked polyvinylpyrrolidone (KOLLIDON® CL-SF)) at the three different amounts, the sweeteners xylitol (XYLITAB® 100) and sucralose, and the lubricant (magnesium stearate) were mixed for a suitable amount of time. Tablets (targeted weight ˜350 mg) were compressed in the Carver Press using an 11 mm die and punch set at a compression of 11.12 KN (˜2500 lbs). The compressed tablets were stored in an air-tight container until further physical tests were performed.

### Crushing Strength, Friability and Disintegration of 3 in 1 Tablets:

The different physical parameters were determined for the 3 in 1 zidovudine/lamivudine/nevirapine tablets. Tablet crushing strength was determined using the Electronik Tablet Hardness tester. Similarly, friability was determined using Roche Friability Tester which was set for 4 minutes at 25 RPM. The disintegration test was performed in 900 mL of simulated salivary fluid maintained at a temperature of 37° C. ±0.2° C.

TABLE 9
3 in 1 Tablet Formulation (Zidovudine/Lamivudine/Nevirapine: 60/30/55 mg)

| | | Amt | | Amt | | Amt | |
|---|---|---|---|---|---|---|---|
| | | per | % w/w | per | % w/w | per | % w/w |
| | | tablet | per | tablet | per | tablet | per |
| Ingredients: | Category | (mg) | tablet | (mg) | tablet | (mg) | tablet |
| Zidovudine | Active | 60.00 | 17.14 | 60.00 | 17.14 | 60.00 | 17.14 |
| Lamivudine | Active | 30.00 | 8.57 | 30.00 | 8.57 | 30.00 | 8.57 |
| Nevirapine | Active | 55.00 | 15.71 | 55.00 | 15.71 | 55.00 | 15.71 |
| LUDIFLASH | Diluent/Disintegrant/ | 159.50 | 45.57 | 159.50 | 45.57 | 159.50 | 45.57 |
| | sweetening agent | | | | | | |
| SSG, CC or | Superdisintegrant | 7.00 | 2.00 | 14.00 | 4.00 | 21.00 | 6.00 |
| KOLLIDON | | | | | | | |
| CLSF | | | | | | | |
| Sucralose | Sweetener | 17.50 | 5.00 | 17.50 | 5.00 | 17.50 | 5.00 |
| Xylitol | Sweetener | 17.50 | 5.00 | 17.50 | 5.00 | 17.50 | 5.00 |
| Magnesium | Lubricant | 3.50 | 1.00 | 3.50 | 1.00 | 3.50 | 1.00 |
| Stearate | | | | | | | |
| Total tablet | | 350.00 | 100.00 | 357.00 | 102.00 | 364.00 | 104.00 |
| weight | | | | | | | |

### Dissolution Studies:

The dissolution study was performed on the formulation with lowest disintegration time with appreciable tablet crushing strength and friability. USP Type 1 method was used, with 900 mL of phosphate buffer, pH 2.0 ±0.02 as the dissolution medium. The basket was set at 100 RPM and the dissolution medium was maintained at 37° C.±0.2° C. Samples were withdrawn at the intervals of 1, 2, 4, 6, 10, 20, 40 and 60 minutes, respectively.

### Physical Parameters of 3 in 1 Tablets for Treating HIV/AIDS in Children

### Disintegration Test, Crushing Strength and Friability:

For the 3-in-1, fixed dose tablets addition of KOLLIDON® CL-SF at the 2% w/w level to the formulation exhibited a suitable disintegration time of 33-39 sec. and a crushing strength of 65-77 N compared to addition of the superdisintegrants at other levels. These values also fall within the acceptable criteria. This formulation was selected for dissolution study (Table 10). In the dissolution study, more than 80% of zidovudine and lamivudine were released within 6 minutes and in around 60 min for the nevirapine. The dissolution profile is presented in FIG. 6. The taste of the tablets was acceptable ranging from 3.0-3.5 on the scale described herein.

TABLE 10
Physical Parameters for 3 in 1 Combination Tablets

| | Level | Average (n = 5) | | |
|---|---|---|---|---|
| Superdisintegrant | (% w/w) | DT (sec) | CS (N) | F (%) |
| SSG | 2 | 71.7 ± 8.0 | 63 ± 8.2 | 1.08 |
| SSG | 4 | 73.7 ± 7.9 | 69 ± 6.5 | 1.68 |
| SSG | 6 | 68.3 ± 6.5 | 63.4 ± 8.4 | 0.99 |
| CC | 2 | 39.7 ± 6.2 | 77.2 ± 10.8 | 0.8 |
| CC | 4 | 35.8 ± 3.7 | 61.6 ± 4.6 | 0.96 |
| CC | 6 | 33.0 ± 2.8 | 65.2 ± 4.9 | 0.84 |
| K-CLSF | 2 | 22.8 ± 2.9 | 74.0 ± 11. 5 | 0.8 |
| K-CLSF | 4 | 25.3 ± 3.1 | 76.4 ± 6.5 | 0.96 |
| K-CLSF | 6 | 33.8 ± 4.4 | 78.8 ± 8.6 | 0.78 |

SSG: Sodium Starch Glycolate; CC: Croscarmellose sodium, K-CLSF: KOLLDDON ® CL-SF DT: Disintegration Time; CS: Crushing Strength; F: Friability (% loss in weight)

### EXAMPLE 4

### Multi-Drug Reconstitutable Granule for Treating HIV/AIDS in Children

The rapidly dissolving multi-drug reconstitutable granule formulations for treating HIV/AIDS in children including the active medicaments lamivudine and zidovudine in the 2 in 1 granules; and lamivudine, zidovudine, and nevirapine in the 3 in 1 granules were made using the following protocol. The 2 in 1 granules and the reconstitutable suspension made from the granules were examined for drug-excipient interaction, stability, dissolution and their drug release profile.

### 2 in 1 Granule Formulation

Sample 2 in 1 granule formulation containing zidovudine and lamivudine were prepared and examined for a variety of chemical and physical properties. The 2 in 1 granule formulations for testing were prepared as follows. The components were mixed by geometric dilution and kneaded using the Hobart mixer for 7 min. The components included: zidovudine and lamivudine as the active medicaments, the excipient, AVICEL® RC 591, as a suspending agent, the one or more sweetening agents included xylitol, sucralose, saccharin and sucrose in various amounts and combinations, parabens preservatives including methyl parabens and/or propyl parabens, and cherry flavorant. The granules were dried at 32° C. overnight and milled using a FITZMILL® Comminutor. The cherry flavor was incorporated into the granules by geometric dilution, the granules allowed to air dry for 12 hrs, and then packaged into amber plastic bottles.

Evaluation of Drug-Excipient Interaction for 2 in 1 Granules: Due to the multi-component nature of multi-drug reconstitutable granule formulations, the potential components, especially the different sweeteners for the suspension were examined for drug-excipient interaction or incompatibility. The components were mixed according to the ratios in which they may be used in the final granule formulation (Table 11). Then 20% w/w of water was added to the powder in glass vials, mixed thoroughly, and stored at 50° C. for three weeks. The powder samples were gently rendered lump free when necessary before analysis. Similarly, identical blends were processed without wetting. The dry blends were stored at room temperature which served as controls. At the end of the storage period, the samples were visually inspected for color and other physical changes. Analytical techniques such as X-ray diffraction, thermogravimetry, high performance liquid chromatography were also used to evaluate drug-excipient interaction or incompatibility. All tests showed no apparent incompatibility, especially when the components were present in the mixtures as shown in Trial nos. 12 or13 that has either two sets of sweeteners—xylitol and sucralose or xylitol and sucrose respectively.

TABLE 11
Combinations of Formulation Components for
Drug-Excipient Characterization

| | Trial Number (amount in % w/v) | | | | | | | |
|---|---|---|---|---|---|---|---|---|
| | 1 | 2 | 3 | 4 | 5 | 6 | 7 | 8 |
| Lamivudine | 1 | 1 | 1 | 1 | 1 | 1 | 1 | 1 |
| Zidovudine | 2.5 | 2.5 | 2.5 | 2.5 | 2.5 | 2.5 | 2.5 | 2.5 |
| AVICEL ® | | 2 | | | | | | |
| Xylitol | | | 40 | | | | | |
| Sucralose | | | | 0.2 | | | | |
| Saccharin | | | | | | | 0.2 | |
| Sucrose | | | | | | | | 40 |
| Methyl paraben | | | | | 0.15 | | | |
| Propyl paraben | | | | | | 0.015 | | |

| | 9 | 10 | 11 | 12 | 13 |
|---|---|---|---|---|---|
| Lamivudine | 1 | 1 | 1 | 1 | 1 |
| Zidovudine | 2.5 | 2.5 | 2.5 | 2.5 | 2.5 |
| AVICEL ® | 2 | 2 | 2 | 2 | 2 |
| Xylitol | 40 | 40 | 40 | 40 | |
| Sucralose | 0.2 | 0.2 | 0.2 | 0.2 | 0.2 |
| Saccharin | | | | | |
| Sucrose | | | | | 40 |
| Methyl paraben | | | 0.15 | 0.15 | 0.15 |
| Propyl paraben | | | | 0.015 | 0.015 |

Stability of 2 in 1 Granules for Reconstitution: Three batches of an optimized granules formulation that contained 2% w/v AVICEL®, 40% w/v xylitol and 0.2% w/v sucralose (Table 12) was chosen for further testing. The ICH Q1F conditions for climatic zones III and IV were used for stability testing (ICH. “Stability testing of new drug substances and products,” Vol. Q1A(R2), International Conference on Harmonisation, 2003). Granules packaged in amber plastic bottles were stored at accelerated stability conditions of 40° C./75% RH for 3 months according to ICH Q1A (ICH. “Stability data package for registration applications in climatic zones III and IV,” Vol. Q1F, International Conference on Harmonisation, 2003). Moisture content, flowability, particle size distribution, content uniformity and other product attributes were evaluated. Degradation products were also evaluated.

TABLE 12
2 in 1 Reconstitutable Granule Formulation
Composition for Suspension Formula

| | Granule | Wt/5 mL | % | Wt/200 mL |
|---|---|---|---|---|
| Ingredient | % (w/w) | (mg) | (w/v) | (mg) |
| Lamivudine | 2.18 | 50 | 1.0 | 2000 |
| Zidovudine | 5.45 | 125 | 2.5 | 5000 |
| AVICEL ® RC591 | 4.36 | 100 | 2 | 4000 |
| Xylitol | 87.21 | 2000 | 40 | 80000 |
| Sucralose | 0.44 | 10 | 0.2 | 400 |
| Methyl paraben | 0.33 | 7.5 | 0.15 | 300 |
| Propyl paraben | 0.03 | 0.75 | 0.015 | 30 |
| Flavorant | 0.0004 | 0.01 | 0.0002 | 0.008 |

Physical Characteristics: The increase in moisture content was not remarkable. The 2 in 1 granules had acceptable flow properties (32-37° angle of repose). The average geometric mean diameter was 325 microns. The mean content uniformity for lamivudine, zidovudine, methyl paraben or propyl paraben was 105.41±1.62%, 106.91+2.29%, 106.33±2.42% and 94.69±3.93% w/w respectively. These met the acceptance criteria. It is important that the content uniformity of the preservatives (e.g., methyl paraben, propyl paraben) be maintained at acceptably high levels as well as the drug content to ensure protection against microbial growth.

Degradation Studies: The granule formulations stored at 40° C./75% RH for 90 days did not show significant degradation. The degradation of methyl paraben and propyl paraben was also not significant after 90 days at the accelerated stability conditions. The potency was maintained for the drugs and preservatives. It was greater than 100% for the active drugs, and greater than 99% and 95% respectively for methyl paraben and propyl paraben respectively during storage.

Dissolution Studies: Percent of the active drug dissolved was more than 80% at 5 min and cumulative release was more than 97% after 90 days of storage (FIG. 7). This meets USP 30 and ICH Q1F criteria set for immediate release zidovudine tablets. The dissolution profiles were similar for the three batches over the 90-day storage, an indication that the manufacturing method was reproducible and the granule formulations were stable.

Reconstituted Suspension from 2 in 1 Granules:

The suspension made from the 2 in 1 granule formulation (Table 12) was evaluated for duration of use (4 weeks) stability, the expected usage time after reconstitution. The reconstituted suspension was equivalent to 10 mg/mL of lamivudine and 25 mg/mL of zidovudine. The suspensions were stored in amber plastic bottles.

Taste Testing: Preliminary taste testing of the liquid suspensions was performed to select the sweetening agents and the concentration range of sweeteners required for effective taste masking. Formulations containing 25% w/v xylitol and 0.2% w/v saccharin or 20% w/v xylitol and 0.3% w/v saccharin were determined to be of palatable taste. Formulations containing 20% w/v xylitol and 0.20% w/v sucralose or 40% xylitol and 0.20% sucralose also had palatable taste. Since 40% w/w xylitol solution also passes BP preservative efficacy test, the formulation that included xylitol and sucralose was selected for further development (Dansico. Xylitol Technical Properties, Danisco Sweeteners Ltd., 2004).

### Effect of Sweeteners on pH and Viscosity of Suspensions of 2 in 1 Granule Formulation:

The effect of the suspending agent and sweetener on the pH and viscosity of the suspensions was examined. AVICEL® RC 591, alone and in combination with other potential formulation components, was suspended in water. The pH of the suspensions was measured using a pH meter. The viscosity of the suspensions was measured on a Brookfield cup and plate RV series viscometer: spindle size 4 at 20 rpm was used. The suspensions were visually inspected for signs of precipitation, sedimentation, and consistency.

Three sample suspensions were prepared for each combination of ingredients. A suspension of AVICEL® RC 591 alone had a pH of 6.74±0.03. The addition of each of the other formulation components, except saccharin, to the AVICEL suspension resulted in minimal reduction in pH ranging from pH 6.42 to 6.69 (Table 13). The addition of saccharin to the AVICEL suspension reduced the pH to 2.37±0.01, which was significant (p<0.05). Sucralose had the least effect on pH (average pH of 6.69±0.01). Formulations containing sucralose or saccharin and all other ingredients including the drugs had pH of 6.72±0.01 and 5.01±0.02, respectively. The low pH produced by saccharin is likely to accelerate the rate of hydrolysis of lamivudine and zidovudine in solution, since both drugs are susceptible to acid hydrolysis. It may also affect viscosity and thus the physical stability of the suspension. Therefore, the effect of saccharin and other components on viscosity was subsequently evaluated.

TABLE 13
Effect of Formulation Components on pH of Suspensions

| | | pH | | | |
|---|---|---|---|---|---|
| Composition | 1 | 2 | 3 | Ave. | SD |
| AVICEL ® | 6.76 | 6.76 | 6.69 | 6.74 | 0.03 |
| Xylitol + A | 6.62 | 6.59 | 6.59 | 6.60 | 0.01 |
| Sucrose + A | 6.47 | 6.51 | 6.51 | 6.50 | 0.02 |
| Sucralose + A | 6.70 | 6.69 | 6.67 | 6.69 | 0.01 |
| Saccharin + A | 2.39 | 2.37 | 2.36 | 2.37 | 0.01 |
| Methyl paraben + Propyl | 6.40 | 6.43 | 6.42 | 6.42 | 0.01 |
| paraben + A | | | | | |
| Xylitol + Sucralose + MP + | 6.70 | 6.73 | 6.72 | 6.72 | 0.01 |
| PP + D + A | | | | | |
| Xylitol + Saccharin + MP + | 4.98 | 5.01 | 5.03 | 5.01 | 0.02 |
| PP + D + A | | | | | |

A = AVICEL ® RC 591, MP = methyl paraben, PP = propyl paraben, D = lamivudine + zidovudine

Viscosity: A suspension of AVICEL® RC 591 alone had an apparent viscosity of 500 centipoise (cP). Suspensions of AVICEL with xylitol, sucrose or methyl paraben/propyl paraben had apparent viscosities of 883.3, 733.3, and 600.0 cP, respectively (Table 14). The two artificial sweeteners sucralose and saccharin reduced viscosity to 416.7 and 0 cP, respectively. The viscosity of the AVICEL suspension decreases with increasing quantity of sucralose. The loss of viscosity of AVICEL in the presence of saccharin is significant (p<0.05).

AVICEL® RC 591 is an anionic colloid of microcrystalline cellulose and sodium carboxymethylcellulose. At low pH, the AVICEL dispersion in water becomes flocculated and loses the linear structure responsible for its viscosity. Since saccharin is acidic in solution, flocculation and a low viscosity were observed. The presence of electrolytes such as Na⁺ also has similar flocculating effect. Although the product specification for AVICEL® RC 591 describes the material to be stable within a pH range of 4-11, under the composition test conditions it was observed to lose its viscosity below pH 5.

A 0.2% w/v solution of sucralose in water has a pH of 6.4, which did not disrupt the colloidal structure of AVICEL dispersion and thus did not cause a significant reduction in AVICEL solution viscosity. Formulations containing the other granule ingredients including the drugs sucralose or saccharin and all had viscosities of 800 and 33.3 cP respectively (Table 14). This was an indication that the effect of saccharin on the pH is more dominant than the effect of all the other formulation components put together. Saccharin was thus excluded from the formulation and sucralose was used for further studies.

TABLE 14
Effect of Formulation Components on Viscosity of Suspensions

| | | Viscosity (cP) | | | |
|---|---|---|---|---|---|
| Composition | 1 | 2 | 3 | Ave. | SD |
| AVICEL ® | 550.0 | 450.0 | 500.0 | 500.0 | 40.8 |
| Xylitol + A | 900.0 | 900.0 | 850.0 | 883.3 | 23.6 |
| Sucrose + A | 750.0 | 750.0 | 700.0 | 733.3 | 23.6 |
| Sucralose + A | 450.0 | 350.0 | 450.0 | 416.7 | 47.1 |
| Saccharin + A | 0.0 | 0.0 | 0.0 | 0.0 | 0.0 |
| MP + PP + A | 650.0 | 550.0 | 600.0 | 600.0 | 40.8 |
| Xylitol + Sucralose + MP + | 850.0 | 800.0 | 750.0 | 800.0 | 40.8 |
| PP + D + A | | | | | |
| Xylitol + Saccharin + MP + | 50.0 | 50.0 | 0.0 | 33.3 | 23.6 |
| PP + D + A | | | | | |

A = AVICEL ® RC 591, MP = methyl paraben, PP = propyl paraben, D = lamivudine + zidovudine

Antimicrobial Effectiveness Testing of Suspensions of 2 in 1 Granule Formulation: The method in USP 30 <51> was used and modified as follows. The 2 in 1 granule formulation (10 g) (Table 12) was suspended in sufficient water to make 100 mL. A volume of the suspension (25 mL) was added to a Petri dish. The plates were inoculated with Escherichia coli and incubated up to 28 days at 35° C. The plates were examined on days 14 and 28 and if growth had occurred, the colonies were counted. Antimicrobial properties of the reconstituted suspensions were maintained for 28 days.

Stability of Reconstituted Suspension of the 2 in 1 Granule Formulation: The reconstituted suspensions of the 2 in 1 granule formulation (Table 12) in amber plastic bottles were stored at long-term storage conditions of 30° C./65% RH for 28 days to simulate in-use conditions. Samples were withdrawn at 7 days intervals for pH, viscosity and potency and sedimentation volume tests.

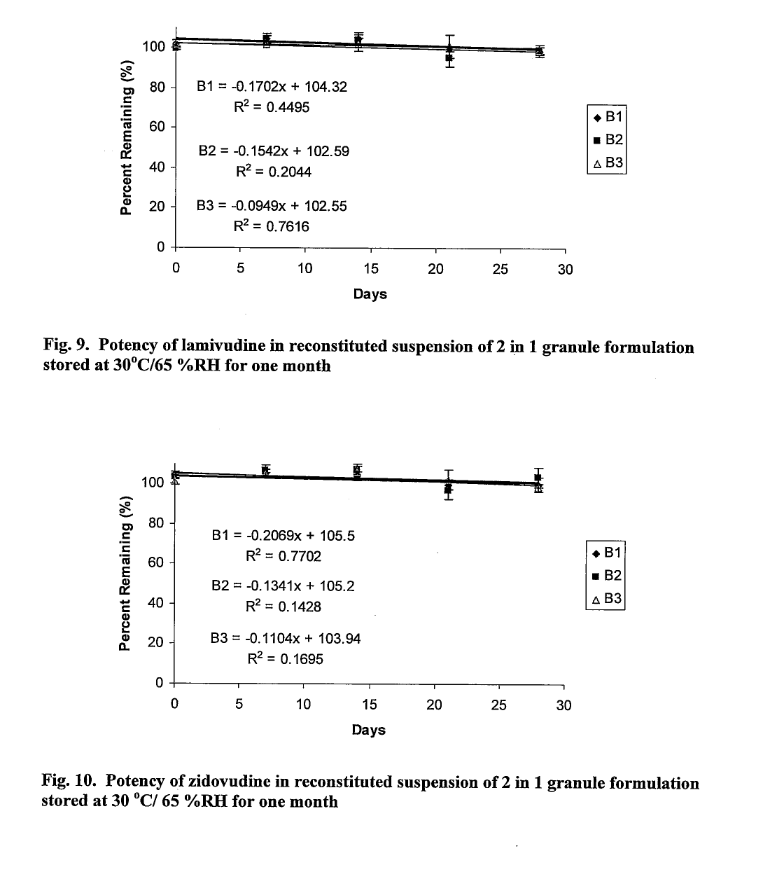

Storage of the reconstituted suspension for 28 days at 30° C./65% RH did not cause changes in the potency, pH and viscosity. There was 98.66% and 99.81% potency of label claim of lamivudine and zidovudine in the reconstituted suspensions, respectively, indicating that the product is stable for use within one month of reconstitution. This is within the acceptable criteria of stability within duration of use (R. G. Strickely et al., “Pediatric drugs—A review of commercially available oral formulations,” J. Pharm. Sci., 2007, 1-44). The estimated shelf-lives for lamivudine and zidovudine in the reconstituted suspensions were 61 days and 51 days respectively. Typical potency profiles of lamivudine and zidovudine in the reconstituted suspension are shown in FIGS. 9 and 10, respectively. Freeze-thaw cycling also showed that the suspension was physically stable. The suspensions did not show any sign of sedimentation. Thus the reconstituted suspension may be used up to 28 days after reconstitution.

### 3 in 1 Granule Formulation

A sample 3 in 1 granule formulation containing zidovudine, lamivudine, and nevirapine were prepared with the composition presented in Table 15. The 3 in 1 granule formulations for testing were prepared as follows. The components were mixed by geometric dilution and kneaded using the Hobart mixer for 7 min. The components included: zidovudine and lamivudine as the active medicaments, the excipient, AVICEL® RC 591, as a suspending agent, the one or more sweetening agents included xylitol and sucralose, and parabens preservatives including methyl parabens and/or propyl parabens. Flavorants including cherry, grape, raspberry, bubble gum, or mixed fruit flavorants, can be added. The granules were dried at 32° C. overnight and milled using a FITZMILL® Comminutor. The flavorant was incorporated into the granules by geometric dilution, the granules were allowed to air dry for 12 hours, and then packaged into amber plastic bottles.

TABLE 15
3 in 1 Reconstitutable Granule Formulation
Composition for Suspension Formula

| | Granule | Wt/5 mL | % | Wt/200 mL |
|---|---|---|---|---|
| Ingredient | % (w/w) | (mg) | (w/v) | (mg) |
| Lamivudine | 1.33 | 30 | 0.6 | 1200 |
| Zidovudine | 2.66 | 60 | 1.2 | 2400 |
| Nevirapine | 2.21 | 50 | 1 | 2000 |
| AVICEL ® RC591 | 4.43 | 100 | 2 | 4000 |
| Xylitol | 88.56 | 2000 | 40 | 80000 |
| Sucralose | 0.44 | 10 | 0.2 | 400 |
| Methyl paraben | 0.33 | 7.5 | 0.15 | 300 |
| Propyl paraben | 0.03 | 0.75 | 0.015 | 30 |
| Flavorant | 0.0004 | 0.01 | 0.0002 | 0.008 |

The drug release profile for the 3 in 1 granule formulation was carried out using USP Type 1 method wherein a USP type 1 basket was used instead of the paddle. A dissolution medium of 900 ml of a phosphate buffer, pH 2.0±0.02 was used for the 3 in 1 granule formulation. Samples (n=6) representing one dose was weighed accurately and transferred in six basket. The baskets were set at 100 RPM and the dissolution medium was maintained at 37° C.±0.2° C. Samples (2 mL) were withdrawn at the intervals of 5, 10, 15, 20, 30, 45 and 60 minutes, due to the relative insolubility of nevirapine in aqueous solution. The dissolution profile for the 3 in 1 granule is shown in FIG. 8.

### EXAMPLE 5

### Granule Particle Size Distribution

The particle size distributions of various granular formulations were determined. As described herein, the granules may be processed using a QUADRO® COMIL® screen mill or a FITZMILL® Comminutor. Other granulation processes may also be utilized. The particle size of the zidovudine granules, the nevirapine granules and the 3 in 1 multi-drug reconstitutable granules (zidovudine, lamivudine, and nevirapine) were determined via sieve analysis. Sieves of different mesh sizes (20, 40, 60, 80, 100, 120, and 200 mesh with the sieve apertures of 850, 425, 250, 180, 150, 125 and 75 microns, respectively) were used. The sieve analysis was performed in a Sieve shaker apparatus (Performer III: Model SS-3) at the amplitude set at “5” for 2 min 30 sec. The results of the particle size analysis are displayed in Table 16.

TABLE 16
Geometric Mean Diameter (GMD) and
Geometric Standard Deviation (GSD)

| Granule Active Ingredient | GMD ( Microns) | GSD |
|---|---|---|
| Zidovudine | 96.21 | 2.33 |
| Nevirapine | 92.64 | 2.32 |
| Zidovudine/Lamivudine/Nevirapine | 299.23 | 2.08 |

### EXAMPLE 6

### Taste for the Granule Formulation

The taste of the granule formulations, the single medicament granule formulation (zidovudine or nevirapine), the 2 in 1 multi-drug granule formulation (zidovudine and lamivudine) and the 3 in 1 multi-drug granule formulation (zidovudine, lamivudine, and nevirapine) were studied using different levels of sweetening agents. The sweeteners were required to mask the bitter flavor associated with the zidovudine and lamivudine medicaments.

Granule formulations having different levels of the sweeteners, sucralose and saccharin, were examined for their ability to increase palatability. A 2×3 design study was implemented using both sweeteners at 3 different levels for both zidovudine and nevirapine single drug formulation (the sweeteners were added separately to each granule formulation). Since saccharin displayed unacceptable viscosity for the reconstituted suspensions of the multi-drug granule formulations, for the multi-drug granule formulation (2 in 1 of zidovudine/lamivudine and 3 in 1 of zidovudine/lamivudine/nevirapine) only sucralose at two different levels was used. The results of the taste studies are presented in Table 17.

TABLE 17
Taste Results for Sweeteners in Granule Formulations

| | Sweetener Concentration (% w/w) | | Taste |
|---|---|---|---|
| Granule Active Ingredient | Sucralose | Saccharin | Score |
| Zidovudine | 0 | 0 | 1 |
| | 2.5 | — | 2 |
| | 3.0 | — | 3 |
| | 3.5 | — | 3.5 |
| | — | 0.2 | 1 |
| | — | 0.5 | 1 |
| | — | 2.5 | 2 |
| Nevirapine | 0 | 0 | 0 |
| | 2.5 | — | 2 |
| | 3.0 | — | 3 |
| | 3.5 | — | 3 |
| | — | 0.2 | 2 |
| | — | 0.5 | 2 |
| | — | 2.5 | 2.5 |
| Zidovudine/Lamivudine | 3.5 | 0 | 2.0 |
| | 5 | 0 | 3.5 |
| Zidovudine/Lamivudine/ | 3.5 | 0 | 2.0 |
| Nevirapine | | | |
| | 5 | 0 | 3.5 |

The taste of the prepared powder mix was then evaluated by two volunteers using the taste score which was assigned from 0-4 scale (0=no taste; 1 =bitter taste; 2=slightly sweet; 3=moderately sweet; 4=very sweet). Based on the good taste scored obtained for each of the physical powder mixtures, the respective sweetener level was selected as the optimum level for each of the four formulations (3.5% sucralose for the zidovudine granule formulation, 3.0% sucralose for the nevirapine granule formulation, and 5% sucralose for the 2 in 1 multi-drug granule formulation (zidovudine and lamivudine) and the 3 in 1 multi-drug granule formulation (zidovudine, lamivudine, and nevirapine).

### EXAMPLE 7

### Wetting Time for Granule Formulation

There is no official method for disintegration/wetting time of single drug granule formulations. A simulated wetting test as reported by Park (J. H. Park, et al., “An alternative to the USP Disintegration Test for orally disintegrating tablets,” Pharmaceutical Technology, 32(8), 2008) which was modified to evaluate the wetting time of the single medicament granules. A WHATMAN® filter paper disk (8 cm) was placed in a Petri dish (8 cm diameter). A volume of 1.75 mL of simulated salivary fluid was applied to the filter paper to soak it enough to simulate the wet surface of the tongue. The granules were weighed (200 mg), equivalent to one dose, and were placed on the filter paper. Immediately, 0.1% w/w FD&C yellow #6 dye solution was allowed to drop on the top of the powder via burette from a height of 5 mm. The time required for the yellow dye solution to diffuse through the granules was noted as the wetting time. For the zidovudine granules, the wetting time for three tests were 7 sec., 10 sec., and 12 sec., for an average wetting time of 9.67±2.52. For the nevirapine granules, the wetting time for three tests were 9 sec., 11 sec., and 12 sec., for an average wetting time of 10.67±1.53.

1. A rapidly dissolving pharmaceutical formulation to reduce the incidence of mother to child transmission of human immunodeficiency virus comprising:
a therapeutically effective amount of an active medicament selected from zidovudine and nevirapine; and
a non-active ingredients matrix comprising:
one or more excipient comprising from 15% to 95% by weight of the total formulation;
one or more superdisintegrant comprising from 1% to 8% by weight of the total formulation;
one or more sweeteners comprising from 0.1% to 11% by weight of the total formulation; and
one or more lubricants comprising from 0.1% to 3% by weight of the total formulation;
wherein the formulation substantially dissolves in an aqueous solution in less than 60 seconds.

2. The pharmaceutical formulation of claim 1, wherein the pharmaceutical formulation is a rapidly dissolving granule formulation and the non-active ingredients matrix comprises:
one or more excipient comprising from 50% to 95% by weight of the total granule formulation;
one or more superdisintegrant comprising from 1% to 4% by weight of the total granule formulation;
one or more sweeteners comprising from 0.1% to 5% by weight of the total granule formulation; and
one or more lubricants comprising from 0.1% to 3% by weight of the total granule formulation,
wherein the granule formulation substantially dissolves in an aqueous solution in less than 40 seconds.

3. The pharmaceutical formulation of claim 2, wherein the therapeutically effective amount of the active medicament is zidovudine in from 4% to 30% by weight of the total granule formulation.

4. The pharmaceutical formulation of claim 1, wherein the therapeutically effective amount of the active medicament is nevirapine in from 2% to 25% by weight of the total granule formulation.

5. The pharmaceutical formulation of claim 2, wherein the one or more sweeteners are selected from the group consisting of saccharin, sucralose, xylitol, sucrose, aspartame, cyclamate, acesulfame potassium, and combinations of any thereof.

6. The pharmaceutical formulation of claim 2, wherein the granules have a geometric mean diameter ranging from 50 microns to 150 microns

7. The pharmaceutical formulation of claim 1, wherein the pharmaceutical formulation is a rapidly dissolving tablet formulation and the non-active ingredients matrix comprises:
one or more excipient comprising from 15% to 75% by weight of the total tablet formulation;
one or more superdisintegrant comprising from 1.5% to 8% by weight of the total tablet formulation;
one or more sweeteners comprising from 2% to 11% by weight of the total tablet formulation; and
one or more lubricants comprising from 0.5% to 3% by weight of the total tablet formulation.

8. The pharmaceutical formulation of claim 7, wherein the therapeutically effective amount of the active medicament is zidovudine in from 5% to 30% by weight of the total tablet formulation.

9. The pharmaceutical formulation of claim 7, wherein the therapeutically effective amount of the active medicament is nevirapine in from 5% to 25% by weight of the total tablet formulation.

10. The pharmaceutical formulation of claim 7, wherein the one or more sweeteners are selected from the group consisting of from 1.5% to 6% by weight of xylitol, from 0.75% to 5% by weight of sucralose, and combinations thereof.

11. The pharmaceutical formulation of claim 1, wherein the one or more superdisintegrant is selected from the group consisting of croscarmellose sodium, sodium starch glycolate, crosslinked polyvinylpyrrolidone, vinylpyrrolidone-vinyl acetate copolymer, and combinations of any thereof

12. The pharmaceutical formulation of claim 1, wherein the one or more lubricants is selected from the group consisting of magnesium stearate, talc, fumed silica, and combinations of any thereof

13. The use of a pharmaceutical formulation comprising: a therapeutically effective amount of an active medicament selected from zidovudine and nevirapine; and a non-active ingredients matrix comprising:
one or more excipient comprising from 15% to 95% by weight of the total formulation;
one or more superdisintegrant comprising from 1% to 8% by weight of the total formulation;
one or more sweeteners comprising from 0.1% to 11% by weight of the total formulation; and
one or more lubricants comprising from 0.1% to 3% by weight of the total formulation,
in the manufacture of a rapidly dissolving formulation for reducing the incidence of mother to child transmission of human immunodeficiency virus.

14. The use of the formulation of claim 13, wherein the formulation is a rapidly dissolving granule formulation and the non-active ingredients matrix comprises:
one or more excipient comprising from 50% to 95% by weight of the total granule formulation;
one or more superdisintegrant comprising from 1% to 4% by weight of the total granule formulation;
one or more sweeteners comprising from 0.1% to 5% by weight of the total granule formulation; and
one or more lubricants comprising from 0.1% to 3% by weight of the total granule formulation,
wherein the granule formulation substantially dissolves in an aqueous solution in less than 40 seconds.

15. The use of the formulation of claim 13, wherein the therapeutically effective amount of the active medicament is zidovudine in from 4% to 30% by weight of the total granule formulation.

16. The use of the formulation of claim 13, wherein the therapeutically effective amount of the active medicament is nevirapine in from 2% to 25% by weight of the total granule formulation.

17. The use of the formulation of any of claim 13, wherein the formulation is administered to a patient ranging from 0 to 18 months of age.

18. The use of the formulation of claim 13, wherein the formulation is a rapidly dissolving tablet formulation and the non-active ingredients matrix comprises:
one or more excipient comprising from 15% to 95% by weight of the total tablet formulation;
one or more superdisintegrant comprising from 1% to 8% by weight of the total tablet formulation;
one or more sweeteners comprising from 0.1% to 11% by weight of the total tablet formulation; and
one or more lubricants comprising from 0.1% to 3% by weight of the total tablet formulation.

19. The use of the formulation of claim 18, wherein the therapeutically effective amount of the active medicament is zidovudine in from 5% to 30% by weight of the total tablet formulation.

20. The use of the formulation of claim 18, wherein the therapeutically effective amount of the active medicament is nevirapine in from 5% to 25% by weight of the total granule formulation.

21. The use of the formulation of claim 18, wherein the formulation is administered to a patient ranging from 6 months to 18 months in age.

22. A rapidly dissolving pharmaceutical formulation for the treatment of HIV/AIDS in children comprising:
lamivudine in from 0.4% to 10% by weight of the total formulation;
zidovudine in from 0.65% to 25% by weight of the total formulation; and
a non-active ingredients matrix comprising:
one or more excipient comprising from 1.0% to 55% by weight of the total formulation;
one or more superdisintegrant comprising from 0% to 8% by weight of the total formulation;
one or more sweeteners comprising from 0.75% to 92.52% by weight of the total formulation;
one or more flavorants comprising from 0% to 0.20% by weight of the total formulation;
one or more lubricants comprising from 0% to 3% by weight of the total formulation; and
one or more parabens preservative comprising from 0% to 0.44% by weight of the total formulation,
wherein the formulation is in the form of a tablet or granules and substantially dissolves or forms a suspension in an aqueous solution in less than 60 seconds.

23. The pharmaceutical formulation of claim 22, wherein the formulation is in the form of a rapidly dissolving tablet ranging from about 200 mg to about 400 mg in weight.

24. The pharmaceutical formulation of claim 22, wherein the zidovudine comprises from 4.5% to 25% by weight of the total tablet formulation, the lamivudine comprises from 2.2% to 10% by weight of the total tablet formulation, the one or more excipient comprise from 12% to 55% by weight of the total tablet formulation, the one or more superdisintegrant comprise from 1.5% to 8% by weight of the total tablet formulation, the one or more sweeteners comprise from 0.75% to 12% by weight of the total tablet formulation, and the one or more lubricants comprising from 0.5% to 3% by weight of the total tablet formulation.

25. The pharmaceutical formulation of claim 22, further comprising nevirapine in from 4% to 20% by weight of the total tablet formulation.

26. The pharmaceutical formulation of claim 22, wherein the one or more sweeteners comprise xylitol in from 1.5% to 6% by weight of the total formulation and sucralose in from 0.75% to 6% by weight of the total formulation and wherein the one or more flavorants are selected from the group consisting of cherry flavorants, grape flavorants, raspberry, bubble gum, and mixed fruit flavorants.

27. The pharmaceutical formulation of claim 22, wherein the formulation is in the form of rapidly dissolving reconstitutable granules having a geometric mean diameter ranging from 200 microns to 400 microns.

28. The pharmaceutical formulation of claim 22, wherein the zidovudine comprises from 0.65% to 6.5% by weight of the total granule formulation, the lamivudine comprises from 0.4% to 2.65% by weight of the total granule formulation, the one or more excipient comprises a suspending agent in from 1.0% to 5.4% by weight of the total granule formulation, the one or more sweetener comprises xylitol in from 25% to 92% by weight and sucralose in from 0.08% to 0.52% by weight of the total granule formulation, the one or more flavorants comprising from 0.0001% to 0.20% by weight of the total formulation, and the one or more parabens preservative comprises methyl paraben in from 0.075% to 0.40% by weight and propyl paraben in from 0.008% to 0.038% by weight of the total granule formulation.

29. The pharmaceutical formulation of claim 22, further comprising nevirapine in from 0.5% to 2.7% by weight of the total granule formulation.

30. A reconstitutable stable aqueous suspension of the rapidly dissolving pharmaceutical formulation of claim 22.

31. The use of a pharmaceutical formulation comprising:
lamivudine in from 0.4% to 10% by weight of the total formulation;
zidovudine in from 0.65% to 25% by weight of the total formulation; and
a non-active ingredients matrix comprising:
one or more excipient comprising from 1.0% to 55% by weight of the total formulation;
one or more superdisintegrant comprising from 0% to 8% by weight of the total formulation;
one or more sweeteners comprising from 0.75% to 92.52% by weight of the total formulation;
one or more flavorants comprising from 0% to 0.20% by weight of the total formulation;
one or more lubricants comprising from 0% to 3% by weight of the total formulation; and
one or more parabens preservative comprising from 0% to 0.44% by weight of the total formulation,
wherein the formulation is in the form of a tablet or granules and substantially dissolves or forms a suspension in an aqueous solution in less than 60 seconds,
in the manufacture of a rapidly dissolving formulation for the treatment of human immunodeficiency virus (HIV) or acquired immune deficiency syndrome (AIDS) in children.

32. The use of the pharmaceutical formulation of claim 31, wherein the formulation further comprises nevirapine in from 0.5% to 20% by weight of the total formulation.

33. The use of the pharmaceutical formulation of claim 31, wherein the pharmaceutical formulation is administered to a patient ranging from 3 months to 16 years of age.

34. The use of the pharmaceutical formulation of claim 31, wherein the pharmaceutical formulation is administered orally to a patient in the form of a tablet or a reconstituted aqueous suspension.
